SEQUENCE LISTING (5 sequences)

SEQ ID NO: 1 - protein, 9 aa, Unknown
  Description of Unknown Virus sequence
HWSYGLRPG

SEQ ID NO: 2 - protein, 17 aa, Morbillivirus
KLIPNASLIENCTKAEL

SEQ ID NO: 3 - protein, 10 aa, Influenza virus
SSLENFRAYV

SEQ ID NO: 4 - protein, 9 aa, Influenza virus
ASNENMETM

SEQ ID NO: 5 - protein, 17 aa, Gallus sp.
ISQAVHAAHAEINEAGR

(19) United States
(12) Patent Application Publication
(10) Pub. No.: US 2011/0262473 A1
(43) Pub. Date: Oct. 27, 2011

(54) SYNTHETIC VACCINE COMPONENT

(75) Inventors: David Charles Jackson, North Balwyn (AU); Weiguang Zeng, Kensington (AU); Kylie Horrocks, Kyabram (AU)

(73) Assignee: THE UNIVERSITY OF MELBOURNE, Parkville (AU)

(21) Appl. No.: 13/002,613

(22) PCT Filed: Jul. 7, 2009
(86) PCT No.: PCT/AU2009/000876

§ 371 Date: Jul. 5, 2011

Related U.S. Application Data
(60) Provisional application No. 61/078,755, filed on Jul. 7, 2008.

(51) Int. Cl. A61K 39/12; A61P 31/12; A61P 37/02; C07K 17/06; A61K 39/00

(52) U.S. Cl. 424/186.1; 424/193.1; 530/328; 424/185.1; 530/313; 530/326; 530/327

(57) ABSTRACT

The present invention relates generally to the field of synthetic vaccines, components thereof and methods for producing same. More particularly, the present invention provides a component of synthetic vaccines and its use in a modular approach to vaccine production.

### FIELD

The present invention relates generally to the field of synthetic vaccines, components thereof and methods for producing same. More particularly, the present invention provides a component of synthetic vaccines and its use in a modular approach to vaccine production.

### BACKGROUND

Bibliographic details of the publications referred to by author in this specification are collected alphabetically at the end of the description.

The development of effective vaccines to combat infectious disease has significantly improved public health. To date vaccination is responsible for global eradication of smallpox as well as vastly diminishing mortality and morbidity associated with many other diseases. Although traditionally used for prophylactic or therapeutic treatment of infectious disease, modern vaccine development now extends to preventing and treating cancer as well as controlling biological processes such as reproduction.

Prophylactic vaccination stimulates the adaptive immune system to produce immunological memory, poised ready to combat infection. The adaptive immune system composes two arms, humoral and cellular immunity. The main effectors of cellular immunity, cytotoxic T lymphocytes (CTLs), play a key role in clearing cells infected with intracellular pathogens, such as viruses, as well as tumor cells. CTLs recognise processed peptide antigens presented on MHC class I molecules that are on all nucleated cells. Humoral immunity involves production of protective antibody by B cells in response to engagement of a particular antigen shape by B cell receptors (BCRs). Both CTLs and B cells require “help” from CD4+ or T helper (T_{H}) cells to fully carry out their functions. T_{H} cells also recognise processed peptide antigens, but in this case those that are associated with MHC class II molecules. Because MHC class II molecules are only found on a subset of specialised cells (called antigen presenting cells [APCs]), which include macrophages and dendritic cells (DC), APCs are an important vaccine target.

Conventional vaccines are often based upon a complete form of the disease-causing agent and involve administration of whole killed or live attenuated organisms or their toxins. Although effective at inducing protective immunity, drawbacks associated with these approaches such as potential reversion of the pathogen to a virulent form and the inability for use in immunocompromised patients, has witnessed development of new vaccine design approaches. These new approaches, which are based on selected portions of the pathogen, include recombinant protein-based, DNA-based and synthetic peptide-based vaccines.

The rationale of synthetic peptide-based vaccines or epitope-based vaccines is centered around the role that peptides play in the immune response. Such vaccines are predicated on the use of antigenic epitope sequences to induce both humoral and cellular immunity following immunization of subjects.

The use of synthetic peptides as vaccines has many advantages over conventional vaccine approaches mostly with regards to safety and manufacturing.

Although synthetic peptide vaccine development has numerous advantages, there are also disadvantages. The length of the peptide sequence necessary for correct folding may be absent from short peptide sequences. As a consequence these peptides may not display the correct conformation required for B cell recognition in vivo. Although an important consideration for vaccines inducing protective humoral immunity, short synthetic peptide sequences have nevertheless been shown to be capable of inducing biologically active antibody.

The activation of T_{H} cells, required for an effective immune response, is another obstacle that needs to be addressed for synthetic peptide-based vaccine design. One traditional approach is to covalently link the epitope of interest to a carrier protein, which provides a source of T_{H} epitopes. However, this often generates an immune response towards the carrier, which is of greater magnitude than that generated to the epitope of interest. This “carrier-induced epitope suppression” can result in very poor antibody titers being raised against the target epitope. Another approach is to identify “promiscuous” T_{H} epitopes, capable of binding to many HLA types in an outbred population. Incorporation of such T_{H} epitopes into vaccine constructs has proven to be very effective at generating T cell “help” by synthetic epitope-based vaccines (Calvo-Calle et al. Journal of Immunology 159(3):1362-73, 1997; Walker et al. Vaccine 25(41):7111-9, 2007).

Another limitation of synthetic peptides is their sometimes poor immunogenicity due to the absence of features required to signal “danger” to the immune system. Administration of peptide-based vaccines with an adjuvant, such as complete Freund's adjuvant (CFA), can overcome this issue although the use of such adjuvants in humans and animals raises concerns of toxicity. To date the only adjuvant licensed for human use, alum which is based on aluminium salts, is not always able to enhance the immunogenicity of vaccine formulations (Davenport et al. J Immunol 100(5):1139-40, 1968). These issues have lead to the development of alternative adjuvants and delivery systems for enhancing the immune response to synthetic peptides. These include ISCOMS (immunostimulating complexes), liposomes, lipopeptides and larger peptide based constructs including multiple antigenic peptide (MAP) systems.

The concept of lipopeptide vaccines arose in the mid 1980's with reports that incorporating lipids into synthetic peptide constructs could enhance their immunogenicity. Many research groups have incorporated different lipid moieties into vaccine constructs, ranging from simple structures based on palmitic acid (BenMohamed et al. European Journal of Immunology 27(5):1242-53, 1997; BenMohamed et al. Infection and Immunity 72(8):4376-84, 2004) to the more complex S-[2,3-bis(palmitoyloxy)propyl]cysteine (Pam₂Cys) [Batzloff et al. Journal of Infectious Disease 194(3):325-30, 2006; Deliyannis et al. European Journal of Immunology 36(3):770-8, 2006; Jackson et al. Proc Natl Acad Sci USA 101 (43):15440-5, 2004], or Pam₃Cys (Zeng et al. Vaccine 18(11-12).1031-9, 2000; Zeng et al. Journal of Peptide Science 2(1):66-72, 1996). These lipopeptide vaccine constructs displayed, in some circumstances, improved immune system stimulation properties in experimental animals. Apart from just acting as adjuvants, lipopeptide vaccines administered intranasally are capable of inducing systemic and mucosal immunity (Batzloff et al. 2006 supra; Deliyannis et al. 2006 supra; BenMohamed et al. European Journal of Immunology 32(8):2274-81, 2002; BenMohamed et al. Immunology 106(1):113-21, 2002), potentially abolishing the need for “needle” vaccine delivery. In addition to this, an HIV lipopeptide vaccine has been demonstrated to be well tolerated in humans during phase 1 clinical trials (Pialoux et al. Aids 15(1):1239-49, 2001).

A limitation in the potential for lipopeptide vaccine use in animals and humans is in their manufacture. Although branched lipopeptide constructs containing Pam₂Cys do display improved solubility which aids in purification during manufacture, the current approach to vaccine construction is far from efficient. Each new vaccine construct is synthesized in toto with the assembly of sometimes difficult sequences requiring considerable expertise.

Controlling the quality of lipopeptide vaccines produced by contiguous synthesis is frequently difficult and results in lower yields.

There is a need to improve the production of lipopeptide-based vaccines.

### SUMMARY

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 1. A sequence listing is provided after the claims.

Vaccine components are provided for use in a modular approach to vaccine production. More particularly, the present invention is directed to a vaccine component comprising a T-helper (T_{H}) epitope and a lipid moiety joined via a linker having at least one free reactive group. The free reactive group is capable of participating in a linking reaction with a target epitope. Hence, the present invention contemplates a method for the generation of a synthetic, self-adjuvanting lipopeptide vaccine using the chemical ligation of particular target epitope to the free reactive site on the linker of a vaccine component. For example, a target epitope is any peptide or any other agent to which an immune response is to be targeted is chemically ligated to the free reactive site on the linker.

The vaccine component of the present invention enables modular lipopeptide vaccines to be constructed en mass. Target epitopes are ligated to the T_{H}/lipid moieties as required via the linker. The present invention enables, therefore, a high throughput approach to the generation of new vaccine constructs.

Accordingly, one aspect of the present invention is directed to a vaccine component comprising a T-helper (T_{H}) epitope, a lipid moiety and a linker wherein the T_{H} epitope is covalently linked to the lipid moiety via the linker and wherein the linker has a free reactive group.

The present invention further contemplates a method of generating a synthetic, self-adjuvanting lipopeptide vaccine construct, the method comprising chemically ligating a target epitope to a free reactive group on a linker to which a T_{H} epitope and a lipid moiety are covalently joined.

Another aspect of the present invention provides a modular synthetic, self-adjuvanting vaccine component comprising a T_{H} epitope and a linker having at least three reactive sites wherein one reactive site is linked to a lipid moiety, and another reactive site is linked to the T_{H} epitope and the third reactive site is capable of participating in a chemical ligation with a target epitope.

A kit is also provided comprising a first compartment adapted to contain a vaccine component comprising a T_{H} epitope, a lipid moiety and a linker wherein the T_{H} epitope is covalently linked to the lipid moiety via the linker and wherein the linker has a free reactive group, a second compartment adapted to received a target epitope; and optionally a third compartment adapted to contain reagents including for chemical ligation of a target epitope. The kit of this aspect of the present invention may further comprise instructions for use.

The present invention is also directed to the use of vaccine component comprising a T_{H} epitope, a lipid moiety and a linker wherein the T_{H} epitope is covalently linked to the lipid moiety via the linker and wherein the linker has a free reactive group, in the manufacture of a synthetic, self-adjuvanting lipopeptide vaccine.

Methods of vaccination using the modular synthetic, self-adjuvanting vaccine also form part of the present invention as are antibodies and immune cells isolated from subjects vaccinated by the lipopeptide vaccine constructs.

The free reactive group on the linker optionally comprises a removable protecting group.

Reference to “ a free reactive group” includes a single or multiple free reactive groups.

A summary of an aspect of modular production of vaccines is provided in FIG. 1.

A list of sequence identifiers used herein is given in Table 1.

TABLE 1
Summary of sequence identifiers

| SEQ ID NO: | Description |
|---|---|
| 1 | Amino acid sequence of B-cell epitope |
| | from LHRH |
| 2 | Amino acid sequence of T_{H} epitope P25 |
| | from fusion protein of morbillivirus |
| 3 | Amino acid sequence of CD8⁺ T cell |
| | determinant from acid polymerases of |
| | influenza virus |
| 4 | Amino acid sequence of CD8⁺ T cell |
| | determinant from nucleoprotein of |
| | influenza virus |
| 5 | Amino acid sequence of T_{H} epitope of |
| | ovalbumin (OT2) |

Abbreviations used herein are provided in Table 2. Single and three letter amino acid abbreviations are defined in Table 3.

TABLE 2
Abbreviations

| Abbreviation | Definition |
|---|---|
| ABTS | 2,2′-amino-bis(3-ethylbenthiazoline-6- |
| | sulfonic acid) |
| ACN | acetonitrile |
| APC | antigen presenting cell |
| Aoa | aminooxyacetic acid |
| ATC | Tris ammonium chloride |
| BCR | B cell receptor |
| Boc | t-butoxycarbonyl |
| Boc-Aoa oSu | Boc-aminooxyacetyl N-hydroxysuccinimide |
| | ester |
| BSA | bovine serum albumin |
| BSA_{X}PBST | PBST with ‘x’ % bovine serum albumin |
| | (v/v) |
| C | carboxy terminus of peptide |
| CFA | complete Freund's adjuvant |
| CTL | cytotoxic T lymphocyte |
| DBU | 1,8-diazabicyclo-[5.4.0] undec-7-ene |
| DC | dendritic cell |
| DCM | dichloromethane |
| Dde | 1-(4,4-diimethyl-2,6-dioxocyclonex-1- |
| | ylidene)ethyl |
| ddH₂O | double distilled water |
| DICI | 1,3-diisopropylcarbodiimide |
| DIPEA | diisopropylethylamine |
| DMAP | dimethylaminopyridine |
| DMF | N,N′-dimethylformamide |
| DTDP | 2,2′-dithiodipyridine |
| EDTA | ethylenediamine-tetra acetic acid |
| ELISA | enzyme-linked immunosorbent assay |
| ESI | electrospray ionisation |
| FCS | fetal calf serum |
| Fmoc | 9-fluorenylmethoxycarbonyl |
| HBTU | O-benzotriazole-N,N,N′,N′-tetramethyl- |
| | uronium-hexafluorophosphate |
| HLA | human leukocyte antigen |
| HOBt | 1-hydroxybenzotriazole |
| H₂0₂ | hydrogen peroxide |
| ICS | intracellular cytokine staining |
| IFN-γ | interferon-gamma |
| IL | interleukin |
| i.n. | intranasal |
| i.p. | intraperitoneal |
| LHRH | luteinizing hormone releasing hormone |
| MAP | multiple antigenic peptide |
| MHC | major histocompatibility complex |
| MS | mass spectrometry |
| Mtt | 4-methyltrityl |
| N | amino terminus of peptide |
| NP | nucleoprotein |
| O.D. | optical density |
| OT2 | T_{H} epitope from ovalbumin |
| PA | acid polymerase |
| Pam₂Cys | S-[2,3-bis(palmitoyloxy)propyl]cysteine |
| Pam₃Cys | tripalmitoyl-S-glyceryl cysteine |
| PAMP | pathogen associated molecular patterns |
| PBS | phosphate-buffered saline |
| PBSN₃ | phosphate-buffered saline containing 0.1% |
| | sodium azide |
| PBST | phosphate-buffered saline containing 0.05% |
| | Tween-20 |
| PFU | plaque forming units |
| PRR | pattern recognition receptor |
| RP-HPLC | reversed-phase high performance liquid |
| | chromatography |
| R_{T} | retention time |
| s.c. | subcutaneous |
| TCR | T cell receptor |
| TFA | trifluoroacetic acid |
| T_{H} | T helper lymphocyte (cell) |
| T_{H} epitope | An epitope on T_{H} cells |
| TIIPS | Triisopropylsilane |
| TLR | toll-like receptor |
| TNBSA | 2,4,6-trinitrobenzenesulfonic acid |
| tp | Thiopyridyl |
| Tris | tris(hydroxymethyl)-aminomethane |

TABLE 3
Single and three letter amino acid abbreviations

| | | |
|---|---|---|
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Trytophan | Trp | W |
| Tyrosine | Tyr | Y |
| Valine | Val | V |

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIG. 1 is a schematic representation of possible arrangements of epitopes and lipids within the branched structures envisaged. The linker molecule here is lysine (K) and X is the third reactive group that can be used to ligate to the target epitope.

FIG. 2 is a schematic representation of a modular approach for the synthesis of lipopeptide vaccine constructs. A. Any target epitope. The epitope is synthesized C→N followed by attachment of a chemical group (Y). B. The Module. The T helper epitope is synthesized and a lysine (K) residue is coupled to the N-terminus. A chemical group (X), with complementary reactivity to the chemical group (Y) attached to the target epitope, is added at the N-terminal lysine. The lipid moiety is then attached to the ε (epsilon) amino group of the lysine, separated by 2 serine (Ser) residues resulting in a branched structure. C. Lipidated modular vaccine construct. The final vaccine construct is formed by chemoselective ligation (dashed arrow) of the module and the target epitope.

FIG. 3 is a schematic representation showing a synthesis strategy for solid phase peptide synthesis of the modules used for the antibody study. A. Step-wise synthesis of the non-lipidated P25 module (Aoa-K-P25). B. Step-wise synthesis of the lipidated P25 module (Aoa-P₂C-P25). The addition of amino acids lysine (K) and serine (Ser) was achieved by coupling free amino acids to the resin in the presence of activators HOBt, HBTU and DIPEA, dissolved in DMF. The Fmoc protecting group was removed by washing twice with 2.5% v/v DBU in DMF for 5 minutes. The Dde group was removed by treatment with 2% v/v hydrazine hydrate in DMF for 10 minutes. The addition of the aminooxyacetyl group (purple dashed box) was achieved by coupling Boc-Aoa-oSu with DIPEA, dissolved in DMF. Cleavage of the peptide from the resin was carried out using Reagent B (88% v/v TFA, 5% v/v Phenol, 5% v/v ddH₂O, and 2% v/v TIIPS).

FIG. 4 is a schematic representation of a synthesis strategy used for assembly of the thiol-based modules used for CTL analysis. A. Step-wise synthesis of the non-lipidated module (Cys-K-OT2). B. Step-wise synthesis of the lipidated module (Cys-P2C-OT2). The addition of amino acids lysine (K), serine (Ser) and cysteine (purple dashed box) were achieved by coupling the free amino acids to the resin in the presence of activators HOBt, HBTU and DIPEA, dissolved in DMF. The Fmoc protecting group was removed by washing twice with 2.5% v/v DBU in DMF for 5 minutes. The Mtt group was removed by washing for 1 hour with 1% v/v TFA in DCM, continually flushing every 5 minutes. Cleavage of the peptide from the resin was carried out using Reagent B (88% v/v TFA, 5% v/v Phenol, 5% v/v ddH₂O, and 2% v/v TIIPS).

FIG. 5 is a schematic representation of assembly of the lipidated modular construct by oxime bond formation. The B cell epitope LHRH was extended to include a serine residue (grey dashed box) at its N-terminus (Ser-LHRH), removed from the solid-phase support and purified. An aldehyde function (blue dashed box) was generated by oxidation of the N-terminally linked serine residue with sodium periodate. Ligation of the P25 lipidated module containing an aminooxyacetyl group (green dashed box) to the aldehyde-bearing LHRH target epitope was then carried out to yield the final product, the oxime linked lipidated modular construct. The red and green boxes, at the bottom of the figure, contain the epitope amino acid sequences.

FIG. 6 is a schematic representation showing assembly of the lipidated modular construct by thioether bond formation. The CTL epitopes (PA and NP) were extended to include a bromoacetyl group (purple dashed box) at their N-terminus by the coupling of bromoacetic acid to the exposed amino group of the N-terminal amino acid. Ligation of the bromoacetyl-CTL epitope to the N-terminal cysteine thiol group of OT2 lipidated module was then carried out to yield the final product, the thioether linked lipidated modular construct. The red and green boxes, at the bottom of the figure, contain the epitope amino acid sequences.

FIG. 7 is a schematic representation showing assembly of the lipidated modular construct by disulphide bond formation. The CTL epitopes, PA and NP, were extended to include a cysteine residue (blue dashed box) at the N-terminal amino group of the peptide sequence and cleaved from the solid-phase support. The cysteinyl-CTL (Cys-CTL) was then reacted with 2,2′-dithioddipyridine (DTDP). DTDP is composed of two thiopyridyl (tp) groups joined by a disulphide bond which when reacted with the Cys-CTL, the thiopyridyl groups are cleaved and activation of the N-terminal cysteine thiol group occurs via disulphide bond formation with a tp group. Ligation of the thiopyridyl-cysteinyl-CTL (tpCys-CTL) epitope to the N-terminal cysteine thiol group of OT2 lipidated module was then carried out to yield the final product, the disulphide linked lipidated modular construct. The red and green boxes, at the bottom of the figure, contain the epitope amino acid sequences.

FIG. 8 is a schematic representation of vaccine constructs used for the antibody study. Oxime linked modular constructs incorporated a T helper (T_{H}) epitope (green box) and a B cell target epitope (red box) ligated by an oxime bond (purple dashed box) to give a C→N→C orientation. The oxime linked lipidated construct differed only to the oxime linked non-lipidated construct by the inclusion of the lipid group Pam₂Cys (blue dashed box). This was carried out by attachment of two serine residues (Ser) and the lipid moiety to the epsilon amino group of a lysine residue (K) positioned between the T_{H} epitope and the oxime bond. The contiguously synthesised construct was assembled from the C-terminal of the B cell target epitope to the N-terminal of the T_{H} epitope, to give a C→N→C→N orientation. Separating the two epitopes with a central lysine residue allowed for attachment of the lipid group. The red and green boxes, at the bottom of the figure, contain the epitope amino acid sequences.

FIG. 9 is a schematic representation showing RP-HPLC analyses of reactants and final product following assembly of the oxime linked lipidated modular construct. The lipidated P25 module elutes (Aoa-P₂C-P25; blue arrow) at 41.7 minutes and diminished in amount over a 2 hour period. The oxime linked lipidated modular construct (LHRH-oxm-P₂C-P25; red arrow) elutes at 40.8 minutes and increases in amount over the same period.

Chromatography was performed using a Vydac Protein C4 column (4.6 mm×250 mm) at 1 ml/min using 0.1% v/v TFA in ddH₂O and 0.1% v/v TFA in ACN as the limit solvent.

FIG. 10 is a schematic representation of RP-HPLC analyses of reactants and final product following assembly of the oxime linked non-lipidated modular construct. The non-lipidated P25 module (Aoa-K-P25; blue arrow) elutes at 25.7 minutes and diminished over a 2 hour period. The oxime linked non-lipidated modular construct (LHRH-oxm-P25; red arrow) elutes at 27 minutes and increased in amount over the 2 hour period. Chromatography was performed in a Vydac Protein C4 column (4.6 mm×250 mm) at 1 ml/min using 0.1% v/v TFA in ddH2O and 0.1% v/v TFA in ACN as the limit solvent.

FIG. 11 is a graphical representation showing Anti-LHRH antibody titre in mice immunised with peptide constructs. Groups of 5 BALB/c mice (6-8 weeks old) were inoculated subcutaneously with 20 nmol of immunogen in saline on day 0 and day 21. Sera were obtained from blood taken 21 days following the primary (1°, closed symbols) and 10 days following the secondary (2°, open symbols) inoculations and used in an ELISA to determine anti-LHRH antibody titres. Individual titres are shown with the horizontal bar representing the mean value for each group. p values were calculated using a one-way ANOVA and are indicated where appropriate.

FIG. 12 is a schematic representation of vaccine constructs used for the CTL study. Modular constructs incorporated a T helper (T_{H}) epitope (green box) and a B cell target epitope (red box) ligated by either a thioether bond (grey dashed box) or disulphide bond (purple dashed box) to give a C→N→N→C orientation. The lipidated modular constructs differed only to the non-lipidated modular constructs by the inclusion of the lipid group Pam₂Cys (blue dashed box). This was carried out by attachment of two serine residues (Ser) and the lipid moiety to the epsilon amino group of a lysine residue (K). The contiguously synthesised construct was assembled from the C-terminal of the B cell target epitope to the N-terminal of the T_{H} epitope, to give a C→N→C→N orientation. Separating the two epitopes with a central lysine residue allowed for attachment of the lipid group. Within the individual red, green and black boxes, at the bottom of the figure, are shown the epitope amino acid sequences.

FIG. 13 is a graphical representation of RP-HPLC analyses of reactants and final product during synthesis of the thioether-linked PA lipidated modular construct. The lipidated OT2 module (Cys-P₂C-OT2) elutes at 40 minutes and reduces in concentration over the 2 hour period. The thioether-linked PA lipidated modular construct (PA-S-P2C-OT2; red arrow) elutes at 40.3 minutes and increases in concentration over the 2 hour period. Chromatography was performed in a Vydac Protein C4 column (4.6 mm×250 mm) at 1 ml/min using 0.1% v/v TFA in ddH2O and 0.1% v/v TFA in ACN as the limit solvent.

FIG. 14 is a graphical representation showing RP-HPLC analyses of reactants and final product during synthesis of the thioether-linked NP lipidated modular construct. The lipidated OT2 module (Cys-P₂C-OT2) elutes at 40 minutes, reduces in amount and undergoes side reaction (blue arrow) over the 4 hour period shown. The thioether-linked NP lipidated modular construct (NP-S-P₂C-OT2; red arrow) elutes at 40.6 minutes and increases in concentration over the same period. Chromatography was performed in a Vydac Protein C4 column (4.6 mm×250 mm) at 1 ml/min using 0.1% v/v TFA in ddH₂O and 0.1% v/v TFA in ACN as the limit solvent.

FIG. 15 is graphical representation of RP-HPLC analyses of reactants and final product during synthesis of the PA disulphide-linked lipidated modular construct. The lipidated OT2 module (Cys-P₂C-OT2) elutes at 40 minutes and diminished in amount over the 10 minute period. The disulphide-linked PA lipidated modular construct (PA-SS-P₂C-OT2; red oval) elutes at 39.6 minutes and increases in amount over the same period. Chromatography was preformed in a Vydac Protein C4 column (4.6 mm×250 mm) at 1 ml/min using 0.1% v/v TFA in ddH₂O and 0.1% v/v TFA in ACN as the limit solvent.

FIG. 16 is a graphical representation of RP-HPLC analyses of reactants and final product during synthesis of the disulphide-linked NP lipidated modular construct. The lipidated OT2 module (Cys-P₂C-OT2) elutes at 40 minutes and diminished in amount over the 10 minute period. The disulphide linked NP lipidated modular construct (NP-SS-P₂C-OT2; red oval) elutes at 39 minutes and increases in amount over the same period. Chromatography was performed in a Vydac Protein C4 column (4.6 mm×250 mm) at 1 ml/min using 0.1% v/v TFA in ddH₂O and 0.1% v/v TFA in ACN as the limit solvent.

FIG. 17 is a graphical representation of numbers of PA₂₂₄₋₂₃₆ and NP₃₆₆₋₃₇₄ specific IFNγ⁺CD8⁺ cells induced 7 days following primary inoculation of lipidated modular constructs. 6-8 week old naive C57BL/6 mice where inoculated intranasally with either 25 nmol of immunogen in saline or 10⁴ PFU x31 virus on day 0.7 days later lungs were harvested and single-cell suspensions prepared. Cells were stimulated for 5 hours with 1 μg/ml PA₂₂₄₋₂₃₆ or NP₃₆₆₋₃₇₄ peptide. Cells were then stained for their expression of CD8 and IFNγ and analysed using flow cytometry. A, thioether linked modular constructs. B, disulphide linked modular constructs. All modular non-lipidated constructs failed to induce a detectable PA₂₂₄₋₂₃₆ or NP₃₆₆₋₃₇₄ specific response. “PA+NP lipidated modular mixture” refers to administration 25 nmol of each NP and PA lipidated modular constructs. Data show the mean numbers of IFNγ⁺ CD8⁺ cells±SD for 3 mice. The restimulating peptide is indicated in brackets. Statistical analysis was carried out using a one-way ANOVA and compares each group to the non-lipidated control (*, p<0.05; **, p<0.01; ***, p<0.001).

FIG. 18 is a graphical representation of numbers of PA₂₂₄₋₂₃₆ and NP₃₆₆₋₃₇₄ specific IFNγ⁺CD8⁺ cells induced 7 days following secondary inoculation with thioether-linked modular constructs. 6-8 week old naive C57BL/6 mice where inoculated intranasally with 25 mnol of immunogen in saline on day 0. 21 days later mice were administered a second dose of the same immunogen. The viral control group were primed intraperitoneally with 1×10⁷ PFU PR8 virus on day 0 and challenged intranasally on day 21 with 10⁴ PFU x31 virus. On day 28 lungs were harvested and single-cell suspensions prepared. Cells were stimulated for 5 hours with 1 μg/ml PA₂₂₄₋₂₃₆ or NP₃₆₆₋₃₇₄ peptide. Cells were then stained for their expression of CD8 and IFNγ and analysed using flow cytometry. All modular non-lipidated constructs failed to induce a detectable PA₂₂₄₋₂₃₆ or NP₃₆₆₋₃₇₄ specific response. The ‘PA+NP lipidated modular mixture’ refers to 25 nmol of each NP and PA lipidated modular constructs. Data show the mean numbers of IFNγ⁺CD8⁺ cells±SD for 3 mice. The restimulating peptide is indicated in brackets. Statistical analysis was carried out using a one-way ANOVA and compares each group to the non-lipidated control (*, p<0.05; **,p<0.01;***,p<0.001).

### DETAILED DESCRIPTION

Reference to any prior art in this specification is not, and should not be taken as, an acknowledgment or any form of suggestion that this prior art forms part of the common general knowledge in any country.

As used in the subject specification, the singular forms “a”, “an”, and “the” include plural aspects unless the context clearly indicates otherwise. Thus, for example, reference to “a lipopeptide” includes a single lipopeptide, as well as two or more lipopeptide; reference to “an epitope” includes a single epitope or two or more epitopes; reference to “the invention” includes single or multiple aspects of an invention.

The present invention provides a vaccine component for use in a modular approach to synthetic vaccine production. The vaccine component comprises a linker covalently joining a T_{H} epitope to a lipid moiety wherein the linker comprises at least one free reactive site, optionally capped with a protective group.

Accordingly, one aspect of the present invention is directed to a vaccine component comprising a T_{H} epitope, a lipid moiety and a linker wherein the T_{H} epitope is covalently linked to the lipid moiety via the linker and wherein the linker has a free reactive group.

As indicated above, the “free” reactive group may be capped with a protecting group.

A method is, therefore, provided for assembly of lipopeptide-based vaccines, using a modular approach. This is achieved by preparation of the vaccine in segments or modules that incorporate reactive chemical groups which allow chemoselective ligation to form the final vaccine construct. The method herein uses modules comprising common components of lipopeptide vaccines which allows the potential to act as the basis for assembly of many different lipopeptide vaccines. The vaccines assembled using this modular approach are capable of inducing both humoral and cellular immune responses.

In one embodiment, the modules comprise a vaccine component as defined above and target epitope. Chemical ligation of the target epitope to the vaccine component may be by any convenient means such as via oxime chemistry. In one aspect, incorporation of a commercially available Boc protected aminooxyacetic acid is carried out for introduction of an aminooxyacetyl group into the construct to allow subsequent ligation. The present invention extends, however, to any form of linking chemistry including chemoselective ligation.

Conveniently, the linker is an amino acid or analog thereof or any other agent with at least a tri-functional moiety.

The synthetic vaccine when complete comprises a T_{H} epitope, a target epitope and a lipid moiety, all covalently linked together via the at least tri-functional moiety.

Another aspect of the present invention contemplates a method of generating a synthetic, self-adjuvanting lipopeptide vaccine construct, the method comprising chemically ligating a vaccine component comprising a T_{H} epitope, a lipid moiety and a linker wherein the T_{H} epitope is covalently linked to a lipid moiety via the linker and the linker has a free reactive group, to a target epitope via the free reactive group.

Still another aspect of the present invention relates to a method for generating a synthetic, self-adjuvanting lipopeptide vaccine, the method comprising chemically ligating multiple modules together wherein at least one module comprises a peptide having a T_{H} epitope, another module comprises a peptide having a target epitope and a further module comprises a linker having a lipid moiety attached thereto via one of at least three reactive sites wherein the other of at least two reactive sites links the other two modules together.

Another aspect of the present invention provides a multi-modular synthetic, self-adjuvanting vaccine comprising peptide modules comprising a T_{H} epitope and a linker having at least three reactive sites wherein one reactive site is linked to a lipid moiety, and the at least two other reactive sites link the T_{H} epitope and target epitope.

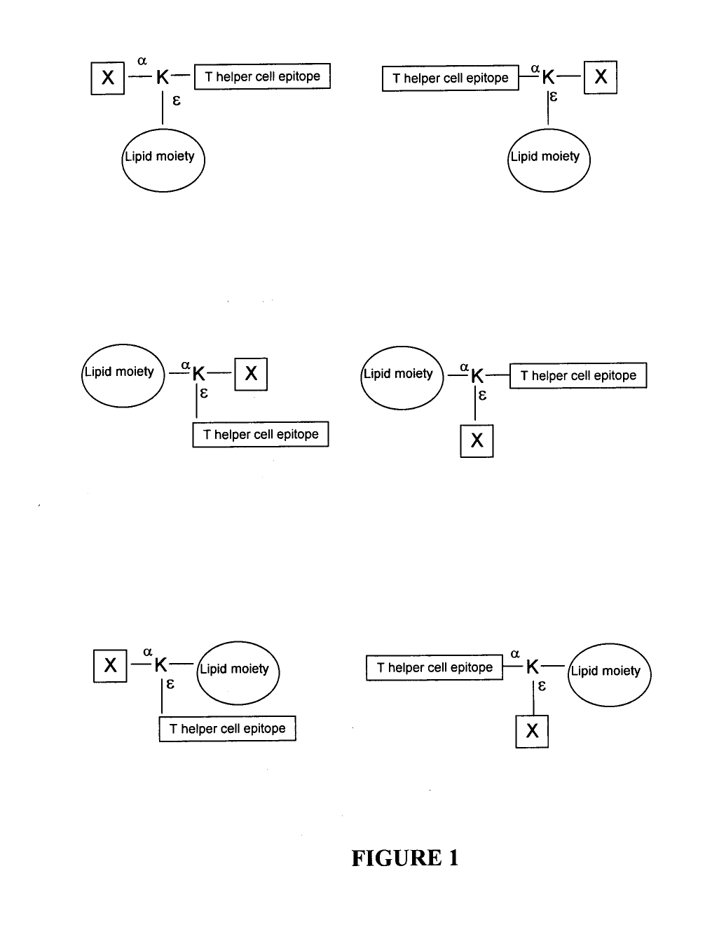

The vaccine components of the present invention are sufficiently immunogenic such that it is generally not necessary to include an extrinsic adjuvant when being used as part of a vaccine. Hence, the vaccine components are referred to herein as “immunogenic components” or “self-adjuvanting molecules” or self-adjuvanting vaccine“. Generalized forms of the vaccine components of the present invention is set forth in FIG. 1.

The immunogenic, multi-modular lipopeptide (or vaccine component) comprises a T_{H} epitope and a lipid moiety covalently linked via reactive sites on a linker which further comprises at least one other free reactive site for use in a chemical ligation to a target epitope moiety. In one embodiment, the lipid moiety comprises a linker component comprising a basic or acidic amino acid. Some basic amino acids used in the present invention have at least two amino groups, such as lysine, ornithine, diaminopropionic acid or diaminobutyric acid. Acidic amino acids have at least two carboxy groups and include aspartic acid or glutamic acid.

As an illustration, the linker (A) may be lysine or a lysine analog, such that the lipid may be attached to either the α or ε group of the lysine. In another embodiment, it is aspartic acid or glutamic acid or an analog thereof.

Use of the epsilon amino group of lysine or the terminal side-chain group of a lysine analog for linkage to the lipid moiety facilitates the synthesis of the peptide moiety as a co-linear amino acid sequence incorporating the target epitope linked to the T_{H} epitope and the lipid moiety via functional reactive site(s) on the linker.

Accordingly, in an embodiment, there is at least one lysine residue or lysine analog to which the lipid moiety is attached to be positioned so as to separate the T_{H} epitope from the reactive group. For example, the lysine residue or lysine analog residue may act as a spacer and/or linking residue between the T_{H} epitope and the reactive group. Naturally, wherein the lysine or lysine analog is positioned between the T-helper epitope and the reactive group, the lipid moiety will be attached at a position that is also between these two, albeit forming a branch from the amino acid sequence of the polypeptide.

The epsilon amino group of the lysine or the terminal side-chain group of a lysine analog can be protected by chemical groups which are orthogonal to those used to protect the alpha-amino and side-chain functional groups of other amino acids. In this way, the epsilon amino group of lysine or the terminal side-chain group of a lysine analog can be selectively exposed to allow attachment of chemical groups, such as lipid-containing moieties, specifically to the epsilon amino group or the terminal side-chain group as appropriate.

The lipid moiety comprises any C₂ to C₃₀ saturated, monounsaturated, or polyunsaturated linear or branched fatty acyl group, or a fatty acid group selected from the group consisting of: palmitoyl, myristoyl, stearoyl, lauroyl, octanoyl, and decanoyl.

In one aspect, the lipid moieties are covalently linked to the T_{H} modules via one of at least 3 reactive sites on a linker. In a related aspect, the lipid moiety is covalently linked via an acidic or basic amino acid positioned between the T_{H} epitope module and, when present, a target epitope module.

Several different fatty acids are known for use in lipid moieties. Exemplary lipids moieties include, without being limited to, palmitoyl, myristoyl, stearoyl and decanoyl groups or, more generally, any C₂ to C₃₀ saturated, monounsaturated, or polyunsaturated fatty acyl group is thought to be useful.

An example of a specific fatty acid moiety the lipoamino acid N-palmitoyl-S-[2,3-bis(palmitoyloxy)propyl]cysteine, also known as Pam₃Cys or Pam₃Cys-OH (Wiesmuller et al. Hoppe Seylers Zur Physiol Chem 364:593, 1983) which is a synthetic version of the N-terminal moiety of Braun's lipoprotein that spans the inner and outer membranes of Gram negative bacteria. Pam₃Cys has the structure of Formula (I):

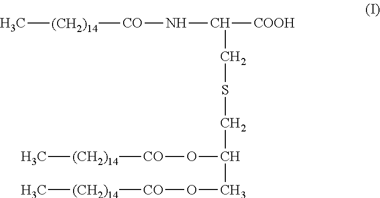

Pam₂Cys (also known as dipalmitoyl-S-glyceryl-cysteine or S-[2,3-bis(palmitoyloxy)propyl]cysteine, an analogue of Pam₃Cys, has been synthesised (Metzger et al. J Pept Sci 1:184, 1995) and been shown to correspond to the lipid moiety of MALP-2, a macrophage-activating lipopeptide isolated from lipidated (Sacht et al. Eur J Immunol 28:4207, 1998; Muhlradt et al. Infect Immun 66:4804, 1998; Muhlradt et al. J Exp Med 185:1951, 1997). Pam₂Cys has the structure of Formula (II):

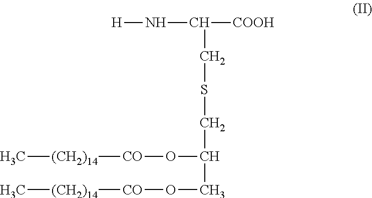

The lipid moiety conjugated to the self-adjuvanting immunogenic molecule of the present invention may be directly or indirectly attached to the linker molecule meaning that they are either juxtaposed in the self-adjuvanting immunogenic molecule (i.e. they are not separated by a spacer molecule) or separated by a spacer comprising one or more carbon-containing molecules, such as, for example, one or more amino acid residues.

The lipid moiety is in a particular embodiment a compound having a structure of general Formula (III):

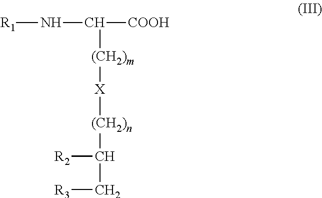

wherein:
(i) X is selected from the group consisting of sulfur, oxygen, disulfide (—S—S—), and methylene (—CH₂—), and amino (—NH—);
(ii) m is an integer being 1 or 2;
(iii) n is an integer from 0 to 5;
(iv) R₁ is selected from the group consisting of hydrogen, carbonyl (—CO—), and R′—CO-wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group;
(v) R₂ is selected from the group consisting of R—CO—O—, R—O—, R—O—CO—, R′—NH—CO—, and R—CO—NH—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group; and
(vi) R₃ is selected from the group consisting of R—CO—O—, R′—O—, R′—O—CO—, R′—NH—O—, and R—CO—NH—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group
and wherein each of R₁, R₂ and R₃ is the same or different.

Depending upon the substituent, the lipid moiety of general Formula (III) may be a chiral molecule, wherein the carbon atoms directly or indirectly covalently bound to integers R₁ and R₂ are asymmetric dextrorotatory or levorotatory (i. e. an R or S) configuration.

In one embodiment, X is sulfur; m and n are both 1; R₁ is selected from the group consisting of hydrogen, and wherein R′ is an alkyl group having 7 to 25 carbon atoms; and R₂ and R₃ are selected from the group consisting of R′—CO—O—, R′—O—, R′—O—CO—, R′—NH—CO—, and R—CO—NH—, wherein R′ is an alkyl group having 7 to 25 carbon atoms.

In a particular embodiment, R′ is selected from the group consisting of: palmitoyl, myristoyl, stearyl and decanol. More preferably, R is palmitoyl.

Each integer R1 in the lipid moiety may be the same or different.

In a particular embodiment, X is sulfur; m and n are both 1; R₁ is hydrogen or R′—CO— wherein R is palmitoyl; and R₂ and R₃ are each R′—CO—O— wherein R is palmitoyl. These particularly preferred compounds are shown by Formula (I) and Formula (II) supra.

Amphipathic molecules, particularly those having a hydrophobicity not exceeding the hydrophobicity of Pam3CyS (Formula (I)) are also contemplated. The lipid moieties of Formula (I), Formula (II), Formula (III) are further modified during synthesis or post-synthetically, by the addition of one or more spacer molecules, preferably a spacer that comprises carbon, and more preferably one or more amino acid residues. These are conveniently added to the lipid structure via the terminal carboxy group in a conventional condensation, addition, substitution, or oxidation reaction. The effect of such spacer molecules is to separate the lipid moiety from the polypeptide moiety and increase immunogenicity of the lipopeptide product.

Serine dimers, trimers, tetramers, etc, are particularly preferred for this purpose.

Exemplary modified lipoamino acids produced according to this embodiment are presented as Formulae (IV) and (V), which are readily derived from Formulae (I) and (II), respectively by the addition of a serine homodimer. As exemplified herein, Pam₃Cys of Formula (I), or Pam₂Cys of Formula (II) is conveniently synthesized as the lipoamino acids Pam₃Cys-Ser-Ser of Formula (IV), or Pam₂Cys-Ser-Ser of Formula (V) for this purpose.

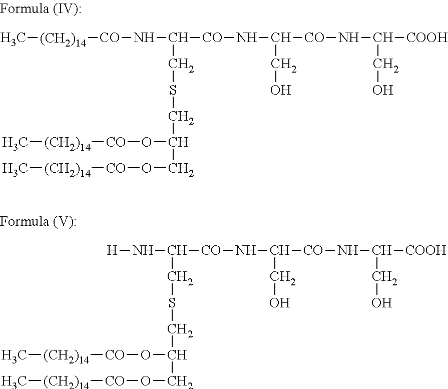

The lipid moiety is prepared by conventional synthetic means, such as, for example, the methods described in U.S. Pat. Nos. 5,700,910 and 6,024,964, or alternatively, the method described by Wiesmuller et al. 1983 supra, Zeng et al. J Pept Sci 2:66, 1996; Jones et al. Xenobiotica 5:155, 1975; or Metzger et al. Int J Pept Protein Res 55:545, 1991). Those skilled in the art will be readily able to modify such methods to achieve the synthesis of a desired lipid for use conjugation to a polypeptide.

Other functional groups such as sulfhydryl, aminooxyacetyl, aldehyde may be introduced into the lipid moieties to enable the lipid moieties to couple to the naturally occurring or recombinant proteins more specifically.

Combinations of different lipids are also contemplated for use in the self-adjuvanting immunogenic molecules of the invention. For example, one or two myristoyl-containing lipids or lipoamino acids are attached via lysine residues to the polypeptide moiety, optionally separated from the polypeptide by a spacer, with one or two palmitoyl-containing lipid or lipoamino acid molecules attached to carboxy terminal lysine amino acid residues. Other combinations are not excluded.

The lipid moiety may comprise any C₂ to C₃₀ saturated, monounsaturated, or polyunsaturated linear or branched fatty acyl group, and preferably a fatty acid group selected from the group consisting of: palmitoyl, myristoyl, stearoyl, lauroyl, octanoyl and decanol. Lipoamino acids are particularly preferred lipid moieties within the present context. As used herein, the term “lipoamino acid” refers to a molecule comprising one or two or three or more lipids covalently attached to an amino acid residue, such as, for example, cysteine or serine, lysine or an analog thereof. In a particular embodiment, the lipoamino acid comprises cysteine and optionally, one or two or more serine residues.

The structure of the lipid moiety is not essential to activity of the resulting self-adjuvanting immunogenic molecule and, as exemplified herein, palmitic acid and/or cholesterol and/or Pam₁CyS and/or Pam₂Cys and/or Pam₃Cys can be used. The present invention clearly contemplates a range of other lipid moieties for use in the self-adjuvanting immunogenic molecules without loss of immunogenicity. Accordingly, the present invention is not to be limited by the structure of the lipid moiety, unless specified otherwise, or the context requires otherwise.

Similarly, the present invention is not to be limited by a requirement for a single lipid moiety unless specified otherwise or the context requires otherwise. The addition of multiple lipid moieties to the naturally molecule is contemplated.

As exemplified herein, highly self-adjuvanting immunogenic lipopeptide molecules capable of inducing T_{H} and/or B cell responses are provided, wherein the self-adjuvanting immunogenic molecule in one aspect comprises Pam₃Cys of Formula (I), or Pam₂Cys of Formula (II) conjugated to the peptide.

The enhanced ability of the self-adjuvanting immunogenic lipopeptides of the present invention to elicit an immune response is reflected by their ability to upregulate the surface expression of MHC class II molecules on immature dendritic cells (DC). In an embodiment, the self-adjuvanting immunogenic lipopeptides are soluble.

Effective lipopeptides are those which are highly soluble. The relative ability of the lipopeptides of the invention to induce an antibody response in the absence of external adjuvant was reflected by their ability to upregulate the surface expression of MHC class II molecules on immature dendritic cells (DC), particularly D1 cells as described by Winzler et al J Exp Med 185, 317, 1997.

In one aspect, the present invention discloses the addition of multiple lipid moieties to the T_{H} epitope.

The positioning of the lipid moiety is selected such that the association of the lipid or moiety does not interfere with the T_{H} or target epitope in such a way as to limit their ability to elicit an immune response. For example, depending on the selection of lipid moiety, the attachment within an epitope may sterically hinder the presentation of the epitope.

As used herein, a T_{H} epitope is any T_{H} epitope which enhances an immune response in a particular target subject (i.e. a human subject, or a specific non-human animal subject such as, for example, a rat, mouse, guinea pig, dog, horse, pig, cow or goat). T_{H} epitopes comprise at least about 10-24 amino acids in length, more generally about 15 to about 20 amino acids in length.

Promiscuous or permissive T_{H} epitopes are contemplated as these are readily synthesized chemically and obviate the need to use longer polypeptides comprising multiple T_{H} epitopes. In related aspects, the T_{H} epitopes selected are those which are able to generate responses across a broad range of HLA types.

Examples of promiscuous or permissive T_{H} epitopes suitable for use in the lipopeptides of the present invention are selected from the group consisting of:
(i) a rodent or human T_{H} epitope of tetanus toxoid peptide (TTP), such as, for example amino acids 830-843 of TTP (Panina-Bordignon et al. Eur J Immun 19: 2237-2242, 1989);
(ii) a rodent or human T_{H} epitope of Plasmodium falciparum pfg27;
(iii) a rodent or human T_{H} epitope of lactate dehydrogenase;
(iv) a rodent or human T_{H} epitope of the envelope protein of HIV or HIVgp120 (Berzofsky et al. J Clin Invest 88:876-884, 1991);
(v) a synthetic human T_{H} epitope (PADRE) predicted from the amino acid sequence of known anchor proteins (Alexander et al. Immunity 1:751-761, 1994);
(vi) a rodent or human T_{H} epitope of measles virus fusion protein (MV-F; Muller et al. Mol Immunol 32:37-47, 1995; Partidos et al. J Gen Virol 71:2099-2105, 1990);
(vii) a T_{H} epitope comprising at least about 10 amino acid residues of canine distemper virus fusion protein (CDV-F) such as, for example, from amino acid positions 148-283 of CDV-F (Ghosh et al. Immunol 104:58-66, 2001; International Patent Publication No. WO 00/46390);
(viii) a human T_{H} epitope derived from the peptide sequence of extracellular tandem repeat domain of MUC1 mucin (US Patent Application No. 0020018806);
(ix) a rodent or human T_{H} epitope of influenza virus haemagglutinin (IV-H) (Jackson et al. Virol 198:613-623, 1994);
(x) a bovine or camel T_{H} epitope of the VP3 protein of foot and mouth disease virus (FMDV-0₁ Kaufbeuren strain), comprising residues 173 to 176 of VP3 or the corresponding amino acids of another strain of FMDV;
(xi) T_{H} epitopes from the fusion protein of the morbillivirus and canine distemper virus (T_{H(MV)});
(xii) T_{H} epitopes from the alpha chain of haemagglutinin of Mem71 influenza virus (T_{H(flu)}); and
(xiii) T_{H} epitopes from chicken ovalbumin (T_{H(ova)}).

As will be known to those skilled in the art, a T_{H} epitope may be recognized by one or more mammals of different species. Accordingly, the designation of any T_{H} epitope herein is not to be considered restrictive with respect to the immune system of the species in which the epitope is recognised. For example, a rodent T_{H} epitope can be recognized by the immune system of a mouse, rat, rabbit, guinea pig, or other rodent, or a human or dog.

The T_{H} epitopes disclosed herein are included for the purposes of exemplification only. Using standard peptide synthesis techniques known to the skilled artisan, the T_{H} epitopes referred to herein are readily substituted for a different T_{H} epitope to adapt the lipopeptide of the invention for use in a different species. Accordingly, additional T_{H} epitopes known to the skilled person to be useful in eliciting or enhancing an immune response in a target species are not to be excluded.

Additional T_{H} epitopes may be identified by a detailed analysis, using in vitro T-cell stimulation techniques of component proteins, protein fragments and peptides to identify appropriate sequences (Goodman and Sercarz Ann Rev Immunol 1:465, 1983; Berzofsky, In: “The Year in Immunology, Vol. 2” page 151, Karger, Basel, 1986; and Livingstone and Fathman Ann Rev Immunol 5:477, 1987).

The peptides may be synthesized by a range of techniques including Fmoc and Boc chemistry. For peptide syntheses using Fmoc chemistry, a suitable orthogonally protected epsilon group of lysine is provided by the modified amino acid residue Fmoc-Lys(Mtt)-OH (NαI-Fmoc-NεM-4-methyltrityl-L-lysine). Similar suitable orthogonally-protected side-chain groups are available for various lysine analogs contemplated herein, eg. Fmoc-Orn(Mtt)-OH (Nα-Fmoc-Nδ-4-methyltrityl-L-Ornithine), Fmoc-Dab(Mtt)-OH (Nα-Fmoc-Nγ-4-methyltrityl-L-diaminobutyric acid) and Fmoc-Dpr(Mtt)-OH (Nα-Fmoc-Nβ-4-methyltrityl-L-diaminopropionic acid). The side-chain protecting group Mtt is stable to conditions under which the Fmoc group present on the alpha amino group of lysine or a lysine analog is removed but can be selectively removed with 1% trifluoroacetic acid in dichloromethane. Fmoc-Lys(Dde)-OH (NI-Fmoc-NM-1-(4,4-dimethyl-2,6-dioxocyclohex-1-ylidene)ethyl-L-lysine) or Fmoc-Lys(ivDde)-OH (NαI-Fmoc-Nε-1-(4,4-dimethyl-2,6-dioxocyclohex-1-ylidene)-3-methylbutyl-L-lysine) can also be used in this context, wherein the Dde side-chain protecting groups is selectively removed during peptide synthesis by treatment with hydrazine.

For peptide syntheses using Boc chemistry, Boc-Lys(Fmoc)-OH can be used. The side-chain protecting group Fmoc can be selectively removed by treatment with piperidine or DBU (1,8-Diazabicyclo[5.4.0]undec-7-ene) but remains in place when the Boc group is removed from the alpha terminus using trifluoroacetic acid.

As indicated above, in certain embodiments, the linker is an acidic or basic amino acid positioned between the T_{H} epitope and target epitope. The lipopeptides of the present invention have the lipid moiety attached to a reactive site on the basic or an acidic amino acid. Basic amino acids have at least two amino groups, such as lysine, ornithine, diaminopropionic acid or diaminobutyric acid. Acidic amino acids have at least two carboxy groups and include aspartic acid or glutamic acid.

Attachment of the lipid moiety can be via the alpha amino group or the terminal amino group of the side-chain of the amino acid residue positioned between the T_{H} epitope and target epitope.

Attachment of the lipid moiety can be via the carboxy group of the amino acid or the terminal carboxy group of the side-chain of the amino acid residue positioned between the T_{H} epitope and target or target epitope.

The present invention is also directed to the use of a first and second peptide modules wherein one module comprises a peptide having a T_{H} epitope and a lipid moiety and another of the modules comprises a peptide having a target epitope, in the manufacture of a synthetic, self-adjuvanting lipopeptide vaccine.

The target epitope is any form of immunogen to which an immune response is to be generated. In certain embodiments, the immunogen is selected from a peptide or small molecule or agent or a protein or a carbohydrate. The immunogens may be specific for inducing a B cell or humoral response i.e. result in the production of antibodies. Alternatively, the immunogen may contain a T cell epitope and induce a cytotoxic T cell response. If the target contains a B cell epitope and a T cell epitope than both types of immune response will arise.

In certain aspects, the target epitope is capable of eliciting the production of antibodies when administered to a mammal when part of the lipopeptide carrier. The antibodies generated bind to the target antigen for which they are specific.

The present invention provides a method of eliciting an antibody response against a target in a subject, the method comprising administering to the subject a synthetic self-adjuvanting lipopeptide vaccine comprising a T_{H} epitope and a lipid moiety and a target epitope, the epitopes and lipid moiety linked via a linker having at least three functional reactive sites.

Another aspect of the present invention provides a method for eliciting an antibody response against a target in a subject, the method comprising administering to a subject a multi-modular synthetic, self-adjuvanting vaccine comprising peptide modules having a T_{H} epitope and a linker having at least three reactive sites wherein one reactive site is linked to a lipid moiety, and the at least two other reactive sites link the T_{H} epitope and target epitope, in which the T_{H} epitope, target epitope and lipid are covalently linked via at least 3 reactive sites on a linker.

The generation of the lipopeptides of the present invention differ in essential aspects from known lipopeptide production techniques in their construction by linking particular modules together. The multi-modular lipopeptides of the present invention have utility in the fields of antibody production, synthetic vaccine preparation, diagnostic methods employing antibodies and antibody ligands, and immunotherapy for veterinary and human medicine.

The effective amount of lipopeptide used in the production of antibodies varies upon the nature of the target epitope, the route of administration, the animal used for immunization, and the nature of the antibody sought. All such variables are empirically determined by art-recognized means.

Reference herein to antibody or antibodies includes whole polyclonal and monoclonal antibodies, and parts thereof, either alone or conjugated with other moieties. Antibody parts include Fab and F(ab)₂ fragments and single chain antibodies. The antibodies may be made in vivo in suitable laboratory animals, or, in the case of engineered antibodies (Single Chain Antibodies or SCAbs, etc) using recombinant DNA techniques in vitro.

In accordance with the present invention, the antibodies may be produced for the purposes of passive immunization of a subject, in which case higher titer or neutralizing antibodies that bind to the target epitope are especially useful.

In accordance with this aspect of the present invention, the antibodies may be produced for the purposes of immunizing the subject, in which case high titer of neutralizing antibodies that bind to the target epitope is especially desired. Suitable subjects for immunization will, of course, depend upon whether the subject is a human to be treated or an animal in order to obtain antibodies for humanization. Non-human animals contemplated herein include, farm animals (e.g. horses, cattle, sheep, pigs, goats, chickens, ducks, turkeys, and the like), laboratory animals (e.g. rats, mice, guinea pigs, rabbits), domestic animals (cats, dogs, birds and the like) and feral or wild exotic animals (e.g. possums, cats, pigs, buffalo, wild dogs and the like).

In another embodiment, monoclonal antibodies according to the present invention are “humanized” monoclonal antibodies, produced by techniques well-known in the art. That is, mouse complementary determining regions (“CDRs”) are transferred from heavy and light V-chains of the mouse Ig into a human V-domain, followed by the replacement of some human residues in the framework regions of their murine counterparts. “Humanized” monoclonal antibodies in accordance with this invention are especially suitable for use in in vivo diagnostic and therapeutic methods. Humanized antibodies include deimmunized antibodies.

Alternatively, the antibodies may be for monitoring purposes to ascertain if a subject has developed antibodies to the target epitope.

By “high titer” means a sufficiently high titer to be suitable for use in diagnostic or therapeutic applications. As will be known in the art, there is some variation in what might be considered “high titer”. For most applications a titer of at least about 10³-10⁴ is considered. More particularly, the antibody titer is in the range from about 10⁴ to about 10⁵, even more particularly in the range from about 10⁵ to about 10⁶.

To generate antibodies, the lipopeptide is optionally formulated with a pharmaceutically acceptable excipient. Administration may be intranasal, intramuscular, sub-cutaneous, intravenous, intradermal, intraperitoneal, or by other known route.

The production of polyclonal antibodies may be monitored by sampling blood of the immunized subject at various points following immunization. A second, booster injection, may be given, if required to achieve a desired antibody titer. The process of boosting and lipidated is repeated until a suitable titer is achieved. When a desired level of immunogenicity is obtained, the immunized subject is bled and the serum isolated and stored, and/or the subject is used to generate monoclonal antibodies (MAbs).

For the production of MAbs any one of a number of well-known techniques may be used, such as, for example, the procedure exemplified in U.S. Pat. No. 4,196,265, incorporated herein by reference.

Any immunoassay may be used to monitor antibody production by the lipopeptide formulations. Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like may also be used.

Alternatively, the target epitope is an immunogen that contains a cytotoxic T lymphocyte (CTL) epitope and induces a cytotoxic CTL response. A CTL epitope can also be defined as an epitope that is recognised by CD8⁺ T cell and includes any epitope which is capable of enhancing or stimulating a CD8⁺ T cell response when administered to a subject. In particular embodiments, the CTL epitopes are at least about 6, 7, 8, 9, 10, 11, 12, 13, 14, 15, 16, 17, 18, 19, 20, 21, 22, 23, 24, 25, 26, 27, 28, 29 or 30 amino acids in length.

For determining the activation of a CTL or precursor CTL or the level of epitope-specific activity, standard methods for assaying the number of CD8⁺ T cells in a specimen can be used. For example, assay formats include a cytotoxicity assay, such as the standard chromium release assay, an assay measuring the production of IFN-γ, such as an ELISPOT assay.

MHC class I tetramer assays can also be utilised, particularly for CTL epitope-specific quantitation of CD8⁺ T cells (Altman et al. Science 274:94-96, 1996; Ogg et al. Curr Opin Immunol 10:393-396, 1998). To produce tetramers, the carboxyl terminus of an MHC molecule, such as the HLA A2 heavy chain, is associated with a specific peptide epitope or polypeptide and treated so as to form a tetramer complex having bound thereto a reporter molecule, such as a fluorochrome (e.g fluoroscein isothiocyanate (FITC), phycoerythrin, phycocyanin or allophycocyanin).

Tetramer formation is achieved, for example, by producing the MHC-peptide fusion protein as a biotinylated molecule and then mixing the biotinylated MHC-peptide with deglycosylated avidin that has been labeled with a fluorophore, at a molar ratio of 4:1. The Tetramers produced bind to a distinct set of CD8⁺ T cell receptors (TCRs) on a subset of CD8⁺ T cells derived from the subject (eg in whole blood or a PBMC sample), to which the peptide is HLA restricted. There is no requirement for in vitro T cell activation or expansion. Following binding, and washing of the T cells to remove unbound or non-specifically bound Tetramer, the number of CD8⁺ cells binding specifically to the HLA-peptide Tetramer is readily quantified by standard flow cytometry methods, such as, for example, using a FACSCalibur Flow cytometer (Becton Dickinson). The Tetramers can also be attached to paramagnetic particles or magnetic beads to facilitate removal of non-specifically bound reporter and cell sorting. Such particles are readily available from commercial sources (e.g. Beckman Coulter, Inc., San Diego, Calif., USA) Tetramer staining does not kill the labeled cells; therefore cell integrity is maintained for further analysis. MHC Tetramers enable the accurate quantitative analyses of specific cellular immune responses, even for extremely rare events that occur at less than 1% of CD8⁺ T cells (Bodinier et al. Nature Med 6:707-710, 2000; Ogg et al. Curr Opin Immunol 10:393-396, 1998).

The total number of CD8⁺ cells in a sample can also be determined readily, such as, for example, by incubating the sample with a monoclonal antibody against CD8 conjugated to a different reporter molecule to that used for detecting the Tetramer. Such antibodies are readily available (eg. Becton Dickinson). The relative intensities of the signals from the two reporter molecules used allows quantification of both the total number of CD8⁺ cells and Tetramer-bound T cells and a determination of the proportion of total T cells bound to the Tetramer.

Because CD4⁺ T-helper cells function in cell mediated immunity (CMI) as producers of cytokines, such as, for example IL-2, to facilitate the expansion of CD8⁺ T cells or to interact with the APC thereby rendering it more competent to activate CD8⁺ T cells, cytokine production is an indirect measure of T cell activation. Accordingly, cytokine assays can also be used to determine the activation of a CTL or precursor CTL or the level of cell mediated immunity in a human subject. In such assays, a cytokine such as, for example, IL-2, is detected or production of a cytokine is determined as an indicator of the level of epitope-specific reactive T cells.

Examples of the cytokine assay format used for determining the level of a cytokine or cytokine production are described by Petrovsky et al. J Immunol Methods 186:37-46, 1995, which assay reference is incorporated herein.

The cytokine assay can be performed on whole blood or PBMC or buffy coat.

By “CMI” is meant that the activated and clonally expanded CTLs are MHC-restricted and specific for a CTL epitope. CTLs are classified based on antigen specificity and MHC restriction, (i.e., non-specific CTLs and antigen-specific, MHC-restricted CTLS). Nonspecific CTLs are composed of various cell types, including NK cells and can function very early in the immune response to decrease pathogen load, while antigen-specific responses are still being established. In contrast, MHC-restricted CTLs achieve optimal activity later than non-specific CTL, generally before antibody production. Antigen-specific CTLs inhibit or reduce the spread of a pathogen and preferably terminate infection.

The self-adjuvanting immunogenic lipopeptide is conveniently formulated in a pharmaceutically acceptable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobial, anti-oxidants, cheating agents and inert gases. The pH and exact concentration of the various components the pharmaceutical composition are adjusted according to routine skills in the art.

The self-adjuvanting immunogenic lipopeptide or derivative or variant or vaccine composition is administered for a time and under conditions sufficient to elicit a humoral response specific for a target antigen.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

The present invention is now described with reference to the Examples provided hereinunder.

The following materials and methods are relevant to the Examples.

Reagents

All reagents, unless otherwise stated, were of analytical grade or equivalent.

Peptide Synthesis

The peptide component(s) of the immunogens were synthesized using solid-phase 9-fluorenylmethoxycarbonyl (Fmoc) chemistry either manually or in a CEM Microwave Peptide Synthesizer (CEM, Matthews, N.C., USA). Constructs containing the B cell target epitope LHRH, which has the sequence HWSYGLRPG [SEQ ID NO:1], and the T_{H} epitope P25 which has the sequence KLIPNASLIENCTKAEL [SEQ ID NO:2] derived from the fusion protein of the morbillivirus, canine distemper virus (Gosh et al. Immunology 104(1):58-66, 2001) were used to study the antibody responses. For investigation of CTL responses constructs incorporated a CD8⁺ T cell determinant (SSLENFRAYV [SEQ ID NO:3]; PA224-236) from either the acid polymerase or the determinant (ASNENMETM [SEQ ID NO:4]; NP366-374) from the nucleoprotein of influenza virus as well as the T_{H} epitope ISQAVHAAHAEINEAGR [SEQ ID NO:5] (OT2) derived from ovalbumin (Robertson et al. Journal of Immunology 164(9):4706-12, 2000; Barnden et al. Immunology and Cell Biology 76(1):34-40, 1998).

For the production of the contiguously synthesized vaccine constructs (P25-P₂C-LHRH and OT2-P₂C-PA), peptides were assembled linearly from the C-terminus of the target epitope to the N-terminus of the T_{H} epitope. For synthesis of the two CTL epitopes and the PA contiguous construct (OT2-P₂C-PA) TentaGel S PHB resin (Wang resin; Rapp Polymere, Tubingen, Germany), to which the first amino acid of the sequence was already attached, with a substitution factor of 0.3 nmol/gram was used, whereas TentaGel S RAM resin (Rapp Polymere) with a substitution factor of 0.23 nmol/gram was used as the solid-phase support for the B cell epitope (LHRH), the LHRH contiguous construct (P25-P₂C-LHRH) and the two T_{H} epitopes (P25 and OT2).

Amino acids purchased from Auspep (Parkville, Australia), Novabiochem (Darmstadt, Germany), or CEM were dissolved in N,N′-dimethylformamide (DMF; Merck, Damstadt, Germany) with a four-fold molar excess of O [symbol] benzotriazole-N,N,N′,N′-tetramethyl-uronium-hexafluorophosphate (HBTU; Novabiochem) and 1-hydroxybenzotriazole (HOBt; CEM) as well as a six-fold molar excess of diisopropylethylamine (DIPEA; Sigma-Aldrich, Steinhiem, Germany). Acylation was carried out for 30 minutes. For manual synthesis of peptides, acylation was monitored using 2,4,6-trinitrobenzenesulfonic acid (TNBSA; Fluka, Buchs, Switzerland) as previously described (Hancock and Battersby Analytical Biochemistry 71(1):260-4, 1976).

Fmoc protecting groups were removed by washing twice with 2.5% v/v 1,6 [symbol] diazabicyclo-[5.4.0]undec-7-ene (DBU; Sigma-Aldrich) in DMF for 5 minutes. Linear amino acid sequences were completed with the addition of a Boc-GlyOH amino acid to the N-terminus of the T_{H} sequence.

Synthesis of Lipidated and Non-Lipidated Modular Constructs

Both lipidated and non-lipidated modular constructs were assembled for the study of both the Ab and CTL responses.

Non-Lipidated P25 Module (Aoa-K-P25) Synthesis

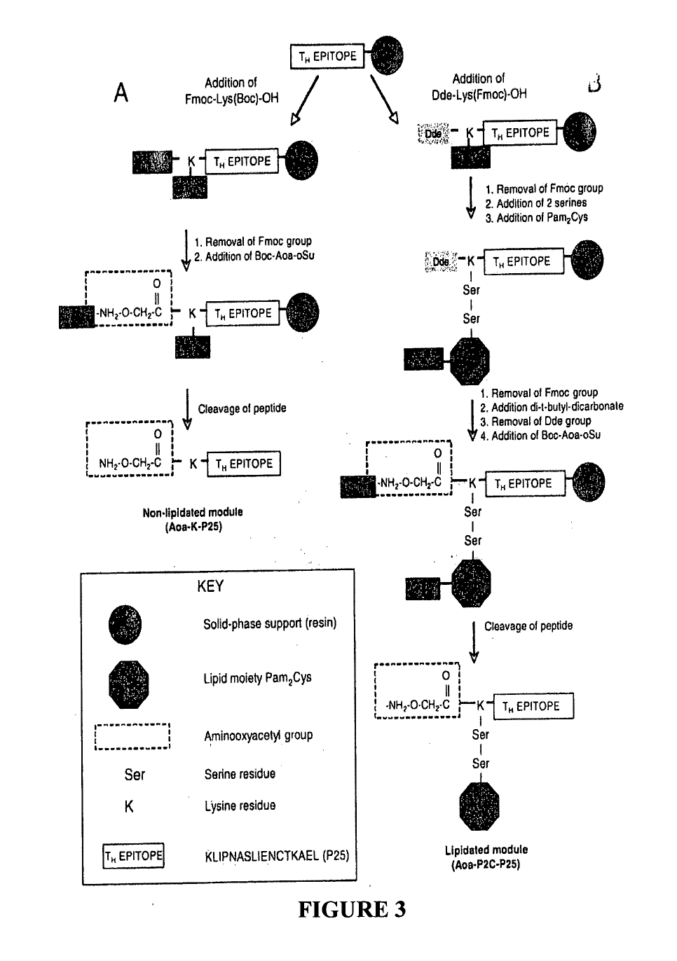

FIG. 3A depicts the step-wise synthesis of this peptide. Following synthesis of the P25 T_{H} epitope, Fmoc-Lys(Boc)-OH was coupled to the exposed a amino group of the N-terminal lysine. Following removal of the Fmoc protecting group Boc-aminooxyacetyl N-hydroxysuccinimide ester (Boc-Aoa-oSu) was then coupled to the exposed α amino group. This was achieved by dissolving a four-fold molar excess of Boc-Aoa-oSu in DMF, adding the solution to the resin and adjusting the pH to 8 with a one-fold molar excess of DIPEA. Coupling was allowed to proceed for 30 minutes after which the resin was washed with DMF. The resin was then washed with acetonitrile (ACN; Merck) and dried under vacuum and then cleaved as described below.

Lipidated P25 Module (Aoa-P₂C-P25) Synthesis

The step-wise synthesis of this peptide is depicted in FIG. 3B. Following synthesis of the P25 T_{H} epitope Dde-Lys(Fmoc)-OH was coupled to the exposed N-terminal amino group of the N-terminal lysine. The Fmoc group protecting the epsilon amino group was then removed and the peptide was lipidated as described below. Once lipidation was complete the Dde (1-(4,4-Dimethyl-2,6 [symbol] dioxocyclohex-1-ylidene)ethyl) group was removed by exposing the resin for 10 minutes to 2% v/v hydrazine hydrate (Fisons, Homebush, Australia) in DMF. Aminooxyacetic acid (Aoa) was then coupled to the exposed α amino group, as described above, providing an aminooxy group for the ligation of the module. The resin was then washed with ACN and dried under vacuum and then cleaved.

Non-Lipidated (Cys-K-OT2) OT2 Module Synthesis

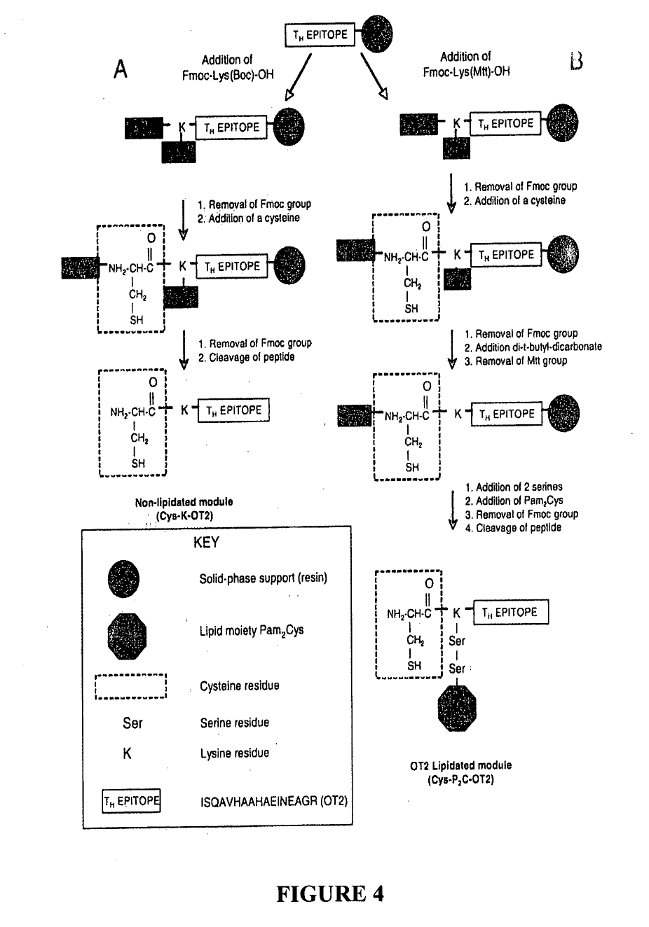

FIG. 4A depicts the step-wise synthesis of this peptide. Following synthesis of the OT2 T_{H} epitope Fmoc-Lys(Boc)-OH was coupled to the exposed a amino group of the N-terminal isoleucine. Following removal of the Fmoc protecting group a cysteine was coupled to the exposed α amino group, the Fmoc group removed and the peptide dried and cleaved as described below.

Lipidated (Cys-P₂C-0T2) OT2 Module Synthesis

The step-wise synthesis of this peptide is depicted in FIG. 4B. Following synthesis of the OT2 T_{H} epitope Fmoc-Lys(Mtt)-OH was coupled to the exposed α amino group of the N-terminal isoleucine. Following removal of the Fmoc protecting group cysteine was coupled to the exposed α amino group to provide a thiol group for ligation with a target epitope. The Fmoc protecting group was then removed. The addition of the more stable Boc (t-butoxycarbonyl) protecting group to the exposed ε amino group of the lysine was achieved by coupling di-t-butyl-dicarbonate (Auspep) to the resin. This was carried out by dissolving a four-fold molar excess di-t-bulyt-dicarbonate in DMF, adding the solution to the resin and adjusting the pH to 8 with equimolar amounts of DIPEA. Coupling was allowed to proceed for 30 minutes after which the resin was washed with DMF and synthesis was continued as described below.

Lipidation

The incorporation of a lysine with an Mtt (4-methyltrityl) or Fmoc protected ε amino group either between the target and T_{H} epitope or between the T_{H} epitope and the orthogonal chemical group of the modular construct (FIG. 4B) provided a point of attachment for Pam₂Cys to yield a branched structure. In the case of Mtt protected amino groups, the Mtt group was removed by subjecting the resin to treatment with 1% v/v trifluoroacetic acid (TFA; Auspep) in dichloromethane (DCM; Merck, Victoria, Australia) for 1 hour, continually washing every 5 minutes. Following removal of the Mtt or Fmoc groups two serine residues were coupled in tandem to the exposed amino group. A four-fold molar excess of Fmoc-Cys-diol and HOBt and a six-fold molar excess of DICI was then dissolved in DMF and the Cys-diol coupled over a period of 1 hour to the terminal branching serine. A 20-fold molar excess of both palmitic acid (Merck, Hohenbrunn, Germany) and 1,3 diisopropylcarbodiimide (DICI; Auspep) and a 2-fold molar excess of dimethylaminopyridine (DMAP; Sigma-Aldrich) in DCM was then added and the reaction allowed to proceed for 16-24 hours, during which time the palmitic acid coupled by esterification to the Cys-diol, following which the resin was washed with DCM. The Fmoc group on the Fmoc-Cys-diol was then removed.

For either continuously synthesised lipopeptides (P25-P₂C-LHRH and OT2-P₂C-PA) or the OT2 lipidated module (Cys-P₂C-OT2) the resin was then dried and cleaved. For synthesis of the P25 lipidated module a Boc protecting group was attached to the exposed amino group by coupling di-t-butyl-dicarbonate as described above. This strategy allowed for synthesis of the remainder of the peptide as outlined above.

Cleavage of Peptide from Solid-Phase Support

To remove the peptide from the solid-phase support and all remaining side chain protecting groups of amino acids the resin was treated with Reagent B (88% v/v TFA, 5% v/v Phenol (BHD Laboratory Supplies, Poole, England), 5% v/v ddH₂O, and 2% v/v triisopropylsilane (TIIPS; Sigma-Aldrich)) for 2-3 hours. Cotton wool filtration was used to separate the peptide from the resin and nitrogen gas was passed over the solution to evaporate excess Reagent B. Diethyl ether (Merck, Kilsyth, Australia) at −20° C. was used to precipitate the peptide which was separated by centrifugation at 4000 rpm for 5 minutes. All peptides were washed in cold diethyl ether air dried and dissolved in equal volumes of ACN and ddH₂O before lyophilization.

Analysis and Purification of Peptide Constructs

All peptides/lipopeptides produced were purified using semi-preparative reversed-phase high performance liquid chromatography (RP-HPLC) with a VYDAC Protein C4 column (10 mm×250 mm; Alltech, NSW, Australia) installed in either a Waters HPLC system (Waters Millipore, Milford, Mass., USA) or a GBC LC HPLC System (GBC Scientific Equipment, Hampshire, Ill., USA). The following linear gradients were used for all lipopeptides: 5 min 0% B, 46 min 82% B, 47 min 100% B where A=ddH₂O with 0.1% v/v TFA and B=ACN with 0.1% v/v TFA. For all non-lipidated peptides, including modified target epitopes, the following gradient was used: 5 min 0% B, 30 min 40% B, 31 min 100% B.

Analytical RP-HPLC was used to determine the homogenicity of peptide and lipopeptide products. The column used was a VYDAC Protein C4 column (4.6 mm×250 mm; Alltech) installed in a Waters HPLC system (Waters Millipore).

Mass analysis of peptides and lipopeptides was performed using electrospray ionisation mass spectrometry (ESI-MS) using the API-electrospray interface of an Agilent 1000 LC/MSD Trap System (Agilent Technologies, Waldbroom, Germany). Bruker Data Analysis 2.1 software (Agilent Technologies) was used to deconvolute the detected charge series for identification of peptides/lipopeptides with a mass/charge of more than 2200.

Production of Lipidated and Non-Lipidated Modular Constructs by Oxime Bond Formation

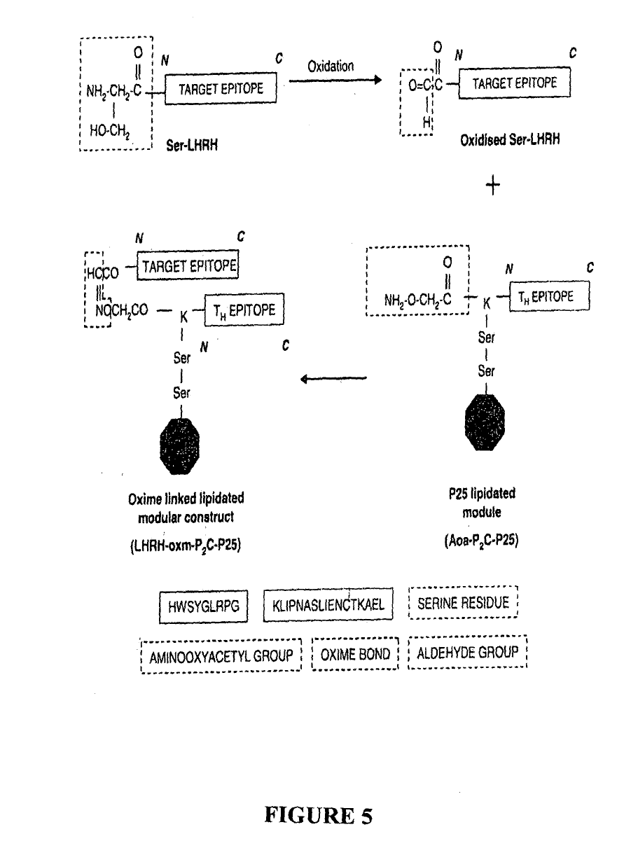

The procedure for assembling the lipidated modular construct by oxime bond formation is depicted in FIG. 5. The B cell epitope LHRH was extended to include a serine residue at the N-terminus. The Ser-LHRH peptide was removed from the resin and purified and an aldehyde functional group created by oxidation of the serine residue with sodium periodate. For this lyophilised Ser-LHRH (1 mg/mi) was dissolved in imidazole buffer (50 Mm, Ph 6.95; ICN Biomedicals, Aurora, Ohio, USA) to which a 2-fold molar excess of sodium periodate (100 Mm in ddH₂O; Sigma-Aldrich, St Louis, Mo., USA) was added. After 5 minutes, the reaction was quenched by adding a 4-fold molar excess of ethylene glycol solution (100 Mm in ddH₂O; Chem Supply, Gillman, South Australia) and the pH adjusted between pH 2-4 by addition of acetic acid (BDH, Poole, England). The oxidized Ser-LHRH was then purified and characterized using MS and RP-HPLC.

Purified LHRH containing an aldehyde group at its N-terminus (1.15 μmol, 1.45 mg) in 2-fold molar excess, was dissolved with either lipidated (0.575 μmol, 1.66 mg) or non-lipidated (0.574 μmol, 1.18 mg) P25 module in 400 μI of 50% v/v ACN, 50% v/v ddH₂O and 0.1% TFA (pH 2). After reaction for 2 hours at room temperature the product LHRH-oxm-P₂C-P25 or LHRH-oxm-P25 was identified and isolated using MS and RP-HPLC.

Production of Lipidated and Non-Lipidated Modular Constructs by Thioether Bond Formation

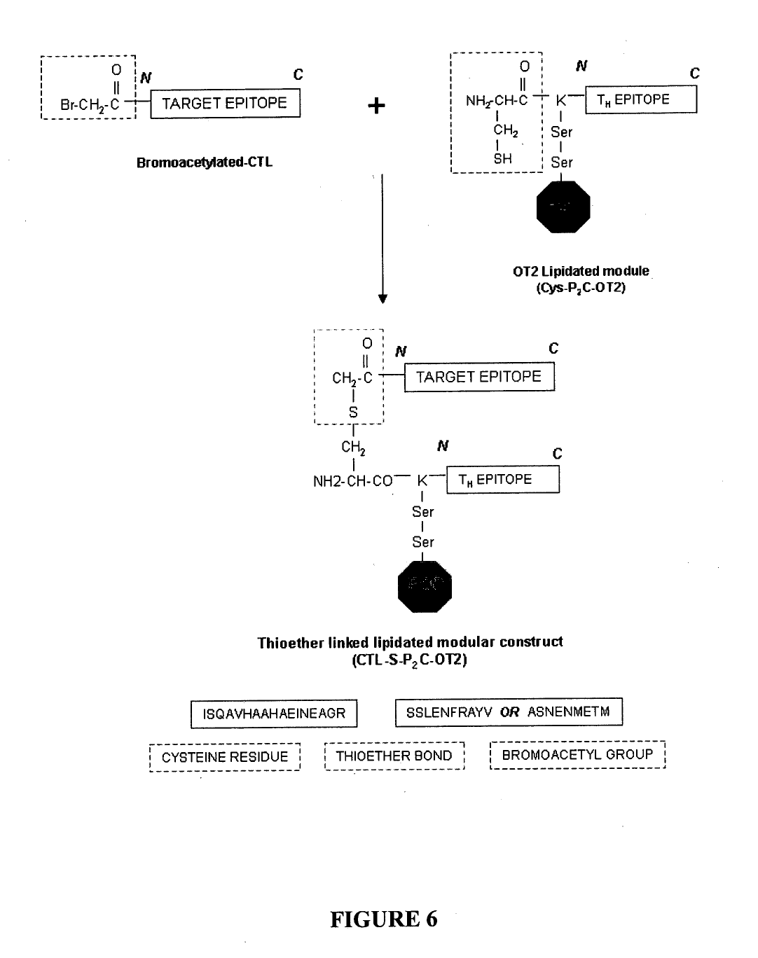

The procedure for assembling the lipidated modular construct by thioether bond formation is displayed in FIG. 6. The PA₂₂₄₋₂₃₆ and NP₃₆₆₋₃₇₄ CTL epitopes were extended to produce BrCH₂CO-PA and BrCH₂CO-NP by coupling bromoacetic acid 99% (Aldrich, Milwaukee, Wis., USA) to the exposed N-terminal amino group. For this a 10-fold molar excess of bromoacetic acid and 5-fold molar excess of DICI was dissolved in DCM and allowed to stand at room temperature for 5 minutes. The solution was then passed through qualitative grade filter paper (Whatman International Ltd., Maidstone, England) and the filtrate added to the resin. After 30 minutes the resin was washed with DCM and dried before cleavage from the resin as described above.

Thioether bond formation between BrCH2CO-CTL and either lipidated or non-lipidated OT2 module was carried out by dissolving both peptides in 400 μM Urea buffer (Ph 4) to which 200 μl pH 8.3 buffer [6M guanidine-hydrochloride (Sigma-Aldrich) in 0.5M Tris(hydroxymethyl)-aminomethane (Tris; Bio-Rad Laboratories, Hercules, Calif., USA), and 2 Mm ethylenediamine-tetra acetic acid (EFTA; Sigma-Aldrich)] was added and allowed to react at room temperature in a light-proof container, BrCH₂CO-PA (2-fold molar excess; 1.0 μmol, 1.31 mg) or BrCH₂CO-NP (10-fold molar excess; 5.0 μmol, 5.73 mg) was added to either purified Cys-P₂C-OT2 (0.5 μmol, 1.42 mg) or Cys-K-OT2 (0.5 μmol, 1.00 mg) and the reactions carried out for 2 hours and 4 hours respectively. The final product CTL-S-P₂C-OT2 or CTL-S-OT2 were identified and isolated from excess BrCH₂CO-CTL by RP-HPLC and MS.

Production of Lipidated and Non-Lipidated Modular Constructs by Disulphide Bond Formation

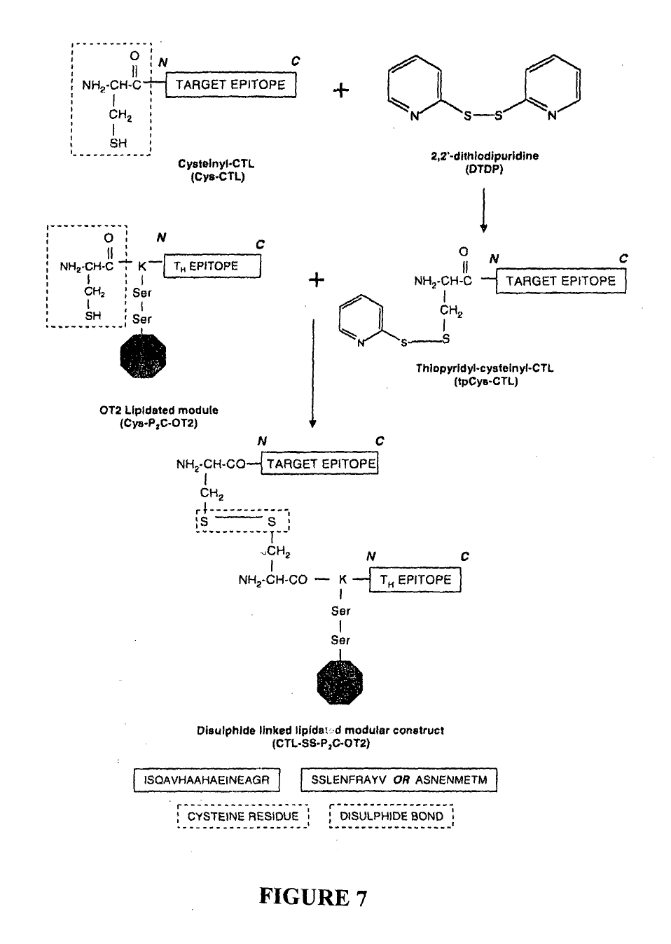

The procedure for assembling the lipidated modular construct by disulphide bond formation is depicted in FIG. 7. The CTL epitopes PA₂₂₄₋₂₃₆ and NP₃₆₆₋₃₇₄ were extended to include a cysteine residue at the N-terminus. The Cys-CTL was then removed from the resin by treatment with Reagent B as described above. Lyophilized Cys-CTL was dissolved in 50% v/v ACN, 50% v/v ddH₂O and 0.1% v/v TFA to which a 0.5-fold molar excess of 2,2′-dithiodipuridine (DTDP; Fluka) dissolved in 100% v/v ACN was added. The reaction was left for 30 minutes at room temperature following which the thiopyridyl-cysteinyl-CTL (tpCys-CTL) epitope was identified and isolated from the remaining DTDP using RP-HPLC and MS.

Either purified Cys-P₂C-OT2 (0.5 μmol, 1.42 mg) or Cys-K-OT2 (0.5 μmol, 1.00 mg) and 2-fold molar excess of tpCys-CTL (tpCys-PA; 1.0 μmol, 1.4 mg or tpCys-NP; 1.0 μmol, 1.23 mg) were reacted in 400 μl of 50% v/v ACN, 50% v/v ddH₂O and 0.1% v/v TFA (pH 2) at room temperature for 10 minutes. The final product CTL-SS-P₂C-OT2 or CTL-SS-OT2 were identified and purified by RP-HPLC and MS.

Immunization and Infection Protocols

BALB/c and C57BL6 mice were bred and housed in the animal facility at the Department of Microbiology and Immunology, The University of Melbourne, Parkville, Australia.

For antibody response studies, groups of five, 6 to 8 week old female BALB/c mice were inoculated subcutaneously (s.c.) in the base of tail with 20 nmol of peptide-based immunogen delivered in 100 μl sterile saline (Media Preparation Facility, The Department of Microbiology and Immunology, The University of Melbourne, Australia) on day O. A negative control group were administered with saline only. Mice were bled and re-inoculated with the same dose of immunogen 21 days following primary inoculation and bled 10 days (day 31) following the secondary inoculation.

For CTL response studies, groups of three, 6 to 8 week old female C57BL/6 mice were anaesthetized by methoxyflurane (Medical Developments International Ltd, Australia) and inoculated intranasally (i.n.) with 25 nmol of the peptide immunogens delivered in 50 μI sterile saline. For study of the primary response, naive mice received either a 25 nmol dose of peptide immunogen or were infected i.n. with 10⁴ PFU influenza virus A/HK-x31 (HKx31, H3N2) in 50 μl phosphate-buffered saline (PBS; Media Preparation Facility). For the study of the secondary response mice received either two 25 nmol doses of peptide immunogen on day 0 and day 21 or were primed intraperitoneally (i.p.) with 10⁷ PFU A/Puerto Rico/8/34 (PR8, HIND in 100 μl PBS and then challenged i.n. 21 days later with 10⁴ PFU HKx31.

Anti-LHRH Enzyme Linked Immunosorbent Assay (ELISA)

Sera were prepared from blood samples and stored at −20° C. until use. ELISAs were as previously described (Brown et al. J Virol 62(1):305-12, 1988). Briefly, flat bottom 96-well polyvinyl plates (Pathtech, Heidelberg West, Victoria, Australia) were coated with 50 μI/well of a solution of 5 μg LHRH/ml in PBS containing 0.1% v/v sodium azide (PBSN₃; i.e. 20 Mm Na₂HPO₄ (Merck, Kilsyth, Australia), 0.15 M NaCl (Ajax Finechem, Auckland, New Zealand), and 0.1% v/v sodium azide (at pH 7.4) and incubated overnight at room temperature (RT) in a humidified container. Excess antigen was removed and 100 μl/well of 10% (w/v) bovine serum albumin in PBS (BSA₁₀PBS; BSA purchased from Sigma-Aldrich) was added for 2 hours. Plates were washed 4 times with PBS containing 0.05% v/v Tween-20 (PBST; Tween-20 purchased from Sigma-Aldrich) and dried over absorbent paper. Serial half-log dilutions of sera were prepared and 50 ml added per well. Following overnight incubation sera was removed and plates washed 6 times with PBST. 50 μl of horseradish peroxidase-conjugated rabbit immunoglobulin directed against mouse immunoglobulin (HRP-RαM; Dako, Denmark) diluted 1/400 in BSA₅PBST was added to each well and incubated for 2 hours. Excess conjugate was discarded and plates washed 6 times with PBST. 100 μl of substrate [1/200 2,2′-amino-bis(3-ethylbenthiazoline-6-sulfonic acid) (ABTS; Sigma-Aldrich) 1/250 hydrogen peroxide (H₂0₂, Merck, Kilsyth, Australia), in 50 Mm citrate buffer (citric acid, Chem Supply) in ddH₂O at pH 4] was added to each well and colour was allowed to develop for 15 minutes. The reaction was stopped with the addition 50 μl of 50 Mm sodium fluoride (BDH Chemicals, Port Fairy, Australia) per well before a Multiskan plate reader (Labsystems, Finland) was used to measure the optical density (O.D.) at both 405 and 450 nm. The antibody titre was determined by expressing the reciprocal logarithmic dilution achieving an O.D. of 0.2, which represents approximately 5 times the background binding in the absence of anti-LHRH antibody.

Tissue Sampling

Lungs taken from mice 7 days following primary or secondary inoculation were cut into pieces using scissors and then subjected to enzymatic digestion with collagenase A (2 mg/mi, Roche, Mannheim, Germany). Following which single-cell suspensions were obtained by pressing lungs through a mesh sieve. Treatment with Tris ammonium chloride (ATC; 7.4% w/v ammonium chloride (Ajax Chemicals), 2.06% w/v Tris, IL ddH₂O) was used to lyse red blood cells. Cell counts were performed using a haemocytometer and cell concentrations were adjusted to 10⁷ cells/ml with RPMI medium (Media Preparation Facility) supplemented with 10% fetal calf serum (FCS; Gibco), 1 Mm sodium pyruvate, 2 Mm L-glutamine, 55 μM 2-mercaptoethanol, 12 mg/ml gentamycin, 100 U/ml penicillin and 100 μg/ml streptomycin (all supplements Gibco), hereafter referred to as RF10.

Intracellular Cytokine Staining for IFN-γ

Cells obtained from lung samples were dispensed into 96-well U-bottomed plates (1×10⁶ cells/well; Nunc, Roskilde, Denmark). Cells were stimulated for 5 hours in 200 μl RF10 containing 1 μl/ml GolgiPlug (BD Biosciences, San Diego, Calif., USA) and 25 U/ml recombinant human IL-2 (Roche) in the presence or absence of 1 μg/ml PA₂₂₄₋₂₃₆ or NP₃₆₆₋₃₇₄ peptides. Cells were then washed with cold FACS buffer (PBS containing 1% v/v FCS and 5 Mm EDTA) and stained with peridinin chlorophyll protein (PerCP) conjugated anti-CD8a-Cy5.5 (BD Pharmingen) on ice for 30 minutes. Following two washes, cells were fixed and permeabilised, using reagents supplied in a Cytoperm/Cytofix kit (BD Biosciences) according to the manufacturer's instructions. After a further two washes, cells were stained for 30 minutes on ice with fluorescein isothiocyanate (FITC) conjugated anti-IFN-γ antibody(BD Pharmingen). After two washes cells were resuspended in FACS buffer and stored at 4° C. Cells were analysed by flow cytometry using a FACSCalibur flow cytometer (Becton Dickinson Immunocytometry Systems) and data were analysed using FlowJo software (version 4.6.2; Tree Star Inc, Ashland, Oreg., USA).

Statistics

All p values were calculated using a one-way ANOVA with a 95% confidence interval using the Tukey test algorithm for post-test analyses.

### EXAMPLE 1

### Synthesis and Immunological Study of Antibody-Inducing Peptide-Based Modular Vaccine Construct

Due to the well characterized response of LHRH and P25 lipopeptides, LHRH was chosen as the target B cell epitope and P25 as the T_{H} epitope for testing the modular methodology. Oxime bond formation was selected as the ligation method due to the presence of a cysteine residue within the T_{H} epitope sequence, which would cause problematic side reactions using thiol-based ligation methods.

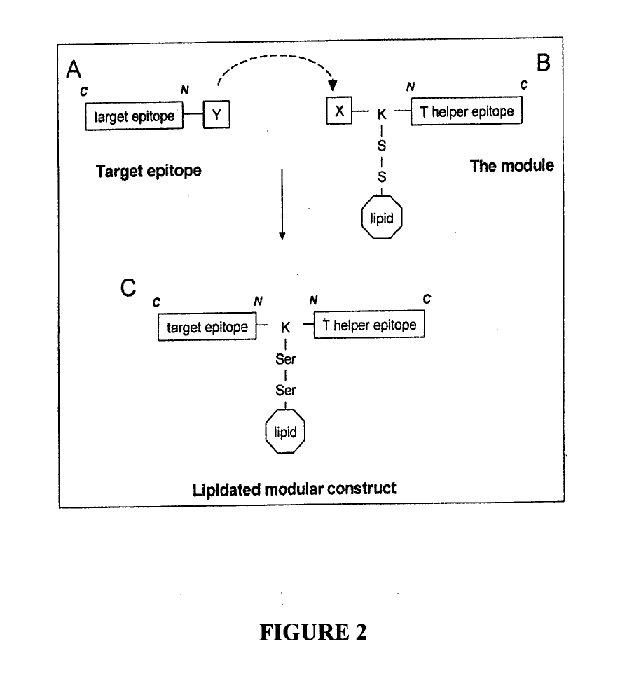

A contiguous vaccine construct, incorporating these epitopes was synthesized to use as a comparison for this new approach. All vaccine constructs were isolated as single species, which possessed the expected mass (Table 4). A generalized scheme for vaccine construct production is shown in FIG. 2.

TABLE 4
Mass of peptides and lipopeptides

| | Theoretical | Determined |
|---|---|---|
| Construct | Mass (Da) | Mass (Da)¹ |
| Ser-LHRH | 1,287.60 | 1,287.09 |
| oxidised Ser-LHRH | 1,256.6 | 1,274 |
| Non-lipidated P25 module | 2,057.20 | 2,058 |
| (Aoa-K-P25) | | |
| Lipidated P25 module | 2,885.2 | 2,885.37 |
| (Aoa-P₂C-P25) | | |
| LHRH contiguous construct | 3.922.85 | 3,922.94 |
| (P25-P₂C-LHRH) | | |
| LHRH oxime linked non- | 3.295.80 | 3.296.69 |
| lipidated construct | | |
| (LHRH-oxm-P25) | | |
| LHRH oxime linked | 4,123.80 | 4,123.01 |
| lipidated construct | | |
| (LHRH-oxm-P2C-P25) | | |

¹Detected using ESI-MS

### EXAMPLE 2

### Development of a New Strategy for Synthesis of the Lipidated Module

A new strategy was developed to allow the aminooxy group to be coupled as the final step before cleavage, thereby avoiding exposure to 1% v/v TFA in DCM (which particularly removes the Boc protecting group of the aminoxy function of Aoa). This was achieved by using Dde-Lys(Fmoc)-OH instead of Fmoc-Lys(Mtt)-OH.

FIG. 3B depicts the synthesis strategy. Briefly this involved addition of Dde-Lys(Fmoc)-OH to the exposed N-terminal amino group of the P25 sequence. The Fmoc protecting group was removed, allowing for addition of two serine residues and the lipid moiety Pam₂Cys. The exposed amino group generated was blocked by a Boc protecting group, as a result of coupling of di-t-butyl-dicarbonate, which inhibited subsequent coupling at this group. The Dde group of the N-terminal lysine was then removed to permit attachment of the aminooxyacetyl group. Boc-Aoa-oSu was choosen for this due to ease of synthesis, because its carboxylic group (COOH) is already activated and only requires DIPEA to be added. RP-HPLC of the cleaved product indicated vastly improved peptide purity and ESI-MS confirmed synthesis of the correct lipopeptide (Table 4).

### EXAMPLE 3

### Synthesis of Oxime Linked Modular Constructs

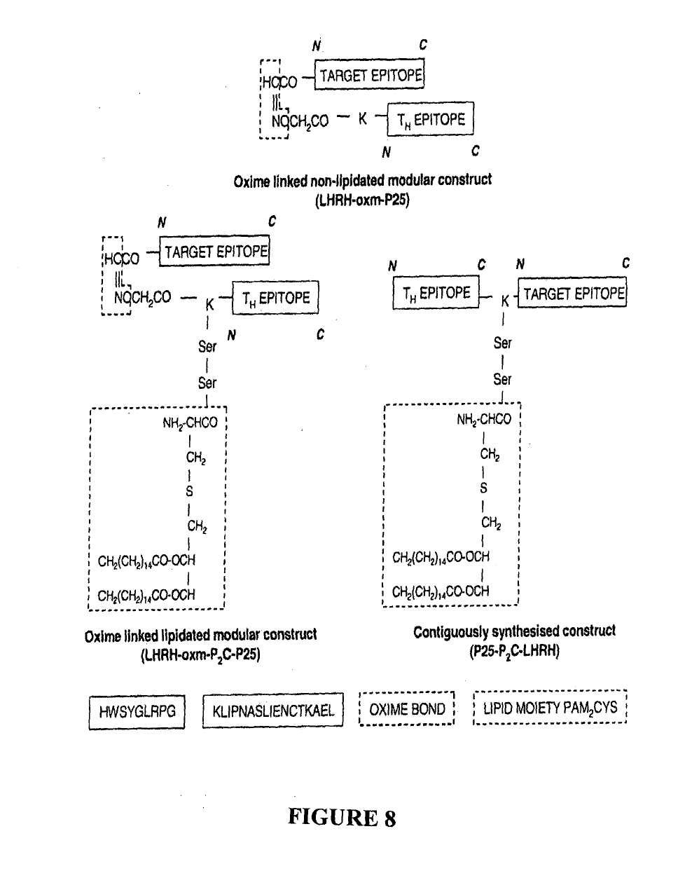

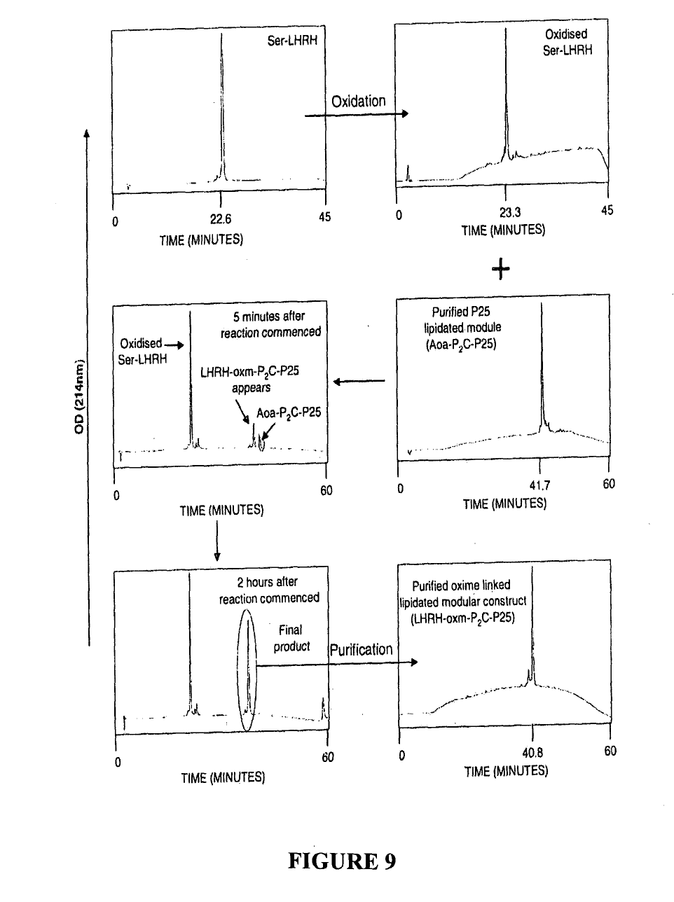

For synthesis of the oxime linked modular vaccine constructs (FIG. 8), the P25 lipidated and non-lipidated modules bearing an aminooxyacetyl group required reaction with an aldehyde group, to form an oxime bond. In order for this to occur an N-terminally linked serine residue was coupled to the LHRH epitope and an aldehyde function generated by oxidation with sodium periodate (FIG. 5). This reaction was complete in 5 minutes and caused a shift in retention time (R_{T}) from 22.6 minutes to 23.3 minutes using RP-HPLC (FIG. 9). The mass of 1274 Da of the product indicated that the hydrated form of the peptide had been produced.

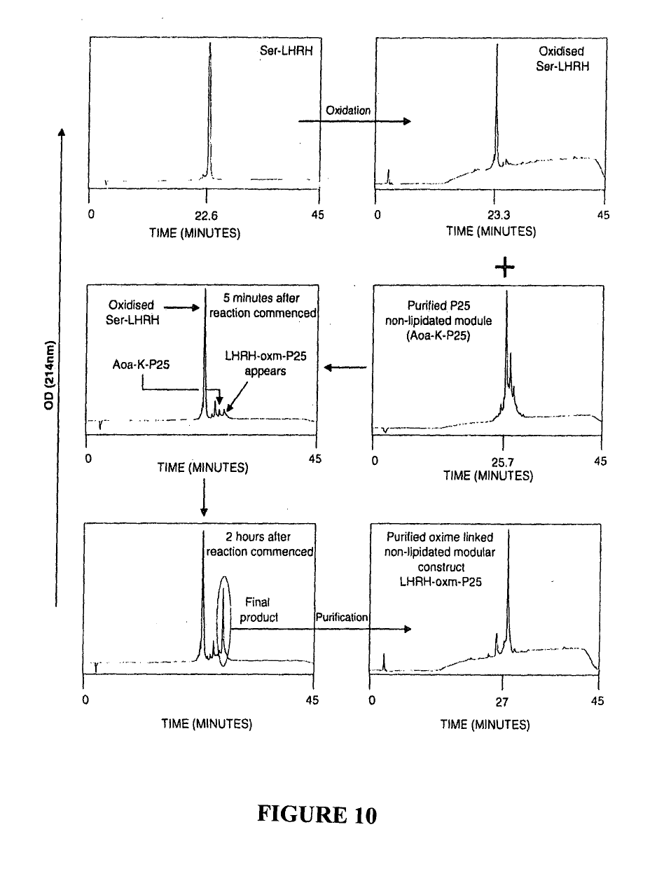

Ligation of the P25 module to the aldehyde-bearing LHRH target epitope took a number of hours to complete. RP-HPLC was used to monitor the reaction at various time points by sampling small volumes of the reaction mixture. The chromatograms of the P25 lipidated module reaction at 5 minutes and 2 hours are shown in FIG. 9, displaying the appearance of the product. The addition of the hydrophilic LHRH sequence to the P25 lipidated module caused a reduction in the overall hydrophobicity of the lipopeptide produced. As the retention time of a molecule is determined by its hydrophobicity within the column, this reduction of hydrophobicity caused the oxime linked lipidated modular construct to elute earlier to that of the P25 lipidated module (FIG. 9). Approximately 5 minutes after initiating the reaction, the peak corresponding to the oxime linked lipopeptide (R_{T} 40.8 min) is larger than the peak corresponding to the P25 lipidated module (R_{T} 41.7 min). However, after two hours of reaction time the amount of the oxime-linked lipidated modular construct had increased and the peak corresponding to the P25 lipidated module was no longer present, indicating that the reaction was complete. Similar results (FIG. 10) were obtained for the non-lipidated oxime construct.

The oxime linked lipidated and non-lipidated modular vaccine constructs were purified using RP-HPLC and analysed using ESI-MS. The purified constructs eluted as single major peaks when analysed using RP-HPLC (final chromatogram, FIGS. 9 and 10) and had the correct mass when examined by ESI-MS (Table 4) indicating that the oxime linked modular constructs produced were of high purity.

### EXAMPLE 4

### The LHRH-Based Modular Vaccine Construct Elicits Strong Anti-LHRH Antibody Response

The immunogenicity of the oxime linked modular constructs, both lipidated and non-lipidated and the contiguously synthesized lipopeptide (P25-P₂C-LHRH), shown previously to induce a strong anti-LHRH antibody response (Chua et al. Vaccine 25:92-101, 2007), were administered in saline to Balb/c mice. The dose of each immunogen administered was 20 nmol for both the primary and secondary inoculation.

An enzyme linked immunosorbent assay (ELISA) detecting serum antibodies directed against LHRH was used to evaluate the antibody response. Mice were bled and re-inoculated, with a similar dose of immunogen, 21 days post-immunisation and bled again on day 31. Mouse sera obtained from both time points, day 21 (primary) and 31 (secondary), were assayed for the presence of anti-LHRH antibodies.

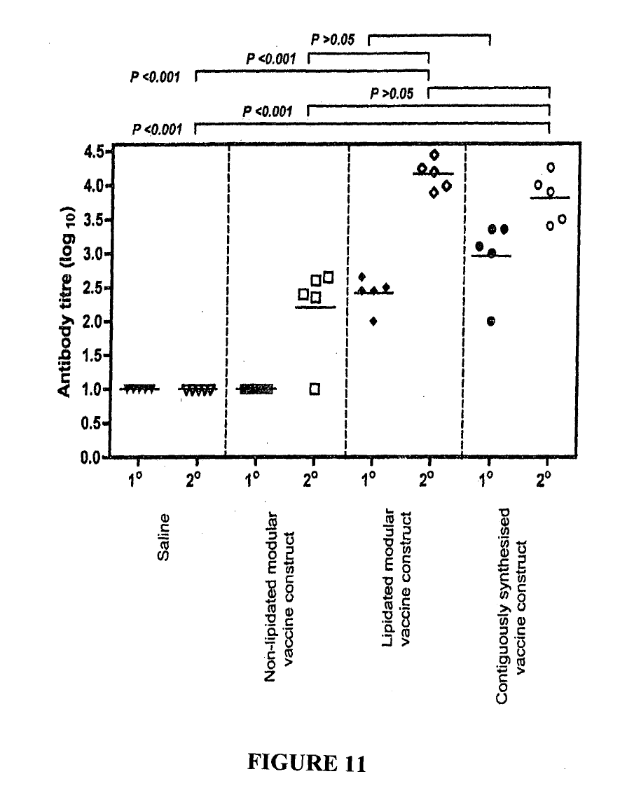

The results (FIG. 11) demonstrate that both the oxime linked lipidated modular construct and the contiguously synthesised construct were able to induce an LHRH specific antibody response following primary inoculation. Although the contiguously synthesised vaccine construct obtained a slightly higher mean anti-LHRH titre, it was not significant in comparison to that produced by the oxime linked lipidated modular construct (p>0.05). The oxime linked non-lipidated modular construct did not appear to elicit a detectable antibody response to LHRH following the primary inoculation.

Following secondary inoculation of the same dose of immunogen, both lipidated vaccine constructs were able to induce an increase in mean anti-LHRH antibody titre (FIG. 11). The oxime linked lipidated modular construct produced a higher secondary anti-LHRH antibody titre than the contiguously synthesised vaccine construct, although this was not significantly different (p>0.05). An increase in the mean anti-LHRH antibody titre was also detected in mice inoculated with the oxime linked non-lipidated modular construct, however, this was determined to be significantly different to the titres induced by both lipidated vaccine constructs (p<0.001).

These results indicate that the different orientation of the B and T_{H} epitopes within the oxime linked lipidated modular construct (viz C→N→N→C) compared with the more usual N→C→N→C orientation of the contiguously synthesised vaccine does not hamper its ability to induce antibody production.

### EXAMPLE 5

### Synthesis and Immunological Study of Peptide-Based Modular Constructs Targeting CTLs

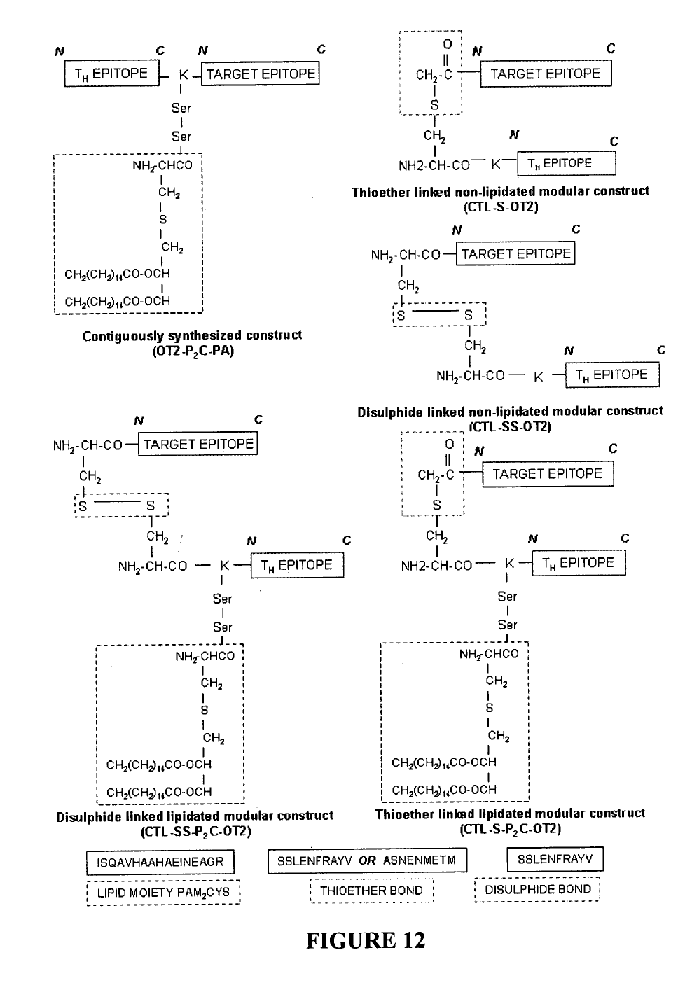

The results herein demonstrated that the novel modular lipidated construct is strongly immunogenic and capable of eliciting a humoral response. It was therefore of interest to determine if this novel methodology could be applied to elicit cellular responses. In this Example, the construction of a modular lipopeptide vaccine is described for targeting CTL. The CTL epitopes chosen for this Example, PA₂₂₄₋₂₃₆ and NP₃₆₆₋₃₇₄, represent two immunodominant CTL epitopes of influenza virus infection in C57BL/6 mice. The T_{H} epitope OT2 derived from ovalbumin, was chosen because it stimulates T_{H} cells in this strain of mouse. The two methionine residues within the NP₃₆₆₋₃₇₄ sequence are prone to oxidation and the use of oxime chemistry to ligate the components of the vaccine was therefore ruled out. Two different chemoselective ligation methods based on thiol chemistry (disulphide and thioether) were therefore examined to assemble these vaccine constructs. The synthetic strategy used for assembly of the thiol-containing lipidated and non-lipidated T_{H} epitope is shown in FIG. 3. The chemical structure of the final disulphide and thioether-based vaccine candidates are shown in FIG. 12.

A contiguous vaccine construct, OT2-P₂C-PA (FIG. 12) was synthesized for use as a comparison in the animal study. Briefly this involved assembly of the T_{H} epitope and PA₂₂₄₋₂₃₆ epitope in a linear form, separated by a lysine residue to which the lipid moiety Pam₂Cys was attached separated by two serine residues. Following cleavage from the solid phase support and purification the PA contiguous construct eluted as a single peak when reanalysed by RP-HPLC. ESI-MS of the collected fraction revealed that the correct peptide construct was obtained (Table 5).

### EXAMPLE 6

### Synthesis of OT2 Modules

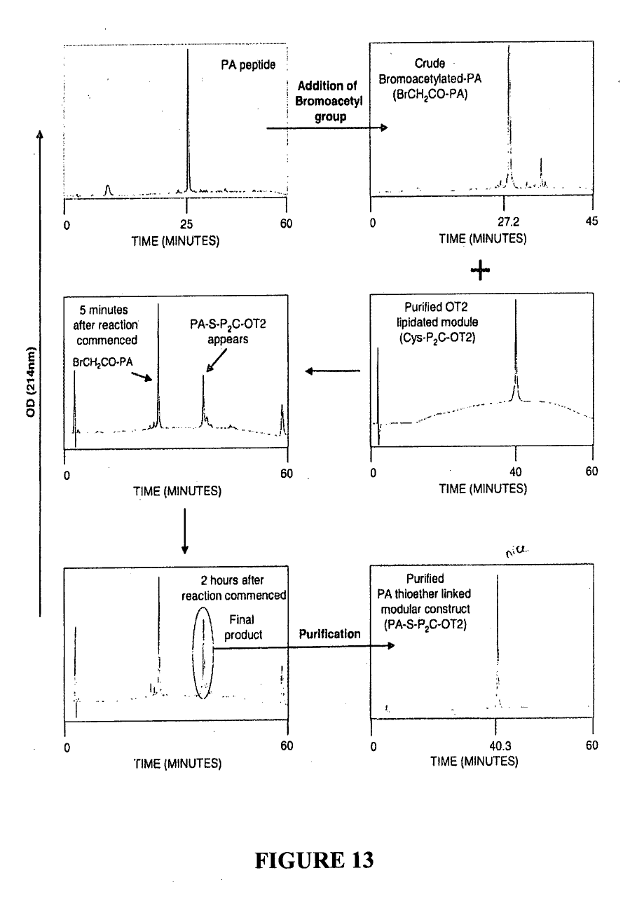

In contrast to synthesis of the P25 lipidated module, preparation of the OT2 lipidated and non-lipidated modules was straightforward. Following removal from the solid-phase support the OT2 modules were purified using RP-HPLC and analysed with ESI-MS. The lipidated OT2 module eluted as a single major peak when analysed by RP-HPLC (FIG. 13) and had the correct mass as determined by ESI-MS (Table 5). The non-lipidated OT2 module also eluted as a single major peak and had the expected mass (Table 5).

TABLE 5
Mass of peptides and lipopeptides used for the CTL study

| | Theoretical | Determined |
|---|---|---|
| Construct | Mass (Da) | Mass (Da)′ |
| PA peptide | 1,184.4 | 1,185.6 |
| NP peptide | 1,025.1 | 1,026.01 |
| Bromoacetyl-PA | 1,306.40 | 1,307.06 |
| Bromoacetyl-NP | 1,147.10 | 1,147.88 |
| Cys-PA | 1,287.60 | 1,288.24 |
| Cys-NP | 1,128.30 | 1,129.1 |
| tpCys-PA | 1,397.60 | 1,397.74 |
| tpCys-NP | 1,238.30 | 1,238.44 |
| OT2 non-lipidated module | 2,002.60 | 2,004.88 |
| (Cys-K-OT2) | | |
| OT2 lipidated module | 2,830.80 | 2,831.34 |
| (Cys-P₂C-OT2) | | |
| PA contiguous construct | 3,952.10 | 3,955.33 |
| (OT2-P₂C-PA) | | |
| PA thioether linked non- | 3,228.10 | 3,230.33 |
| lipidated construct | | |
| (PA-S-OT2) | | |
| NP thioether linked non- | 3,068.8 | 3,070.5 |
| lipidated construct | | |
| (NP-S-OT2) | | |
| PA thioether linked | 4,056.30 | 4,057.24 |
| lipidated construct | | |
| (PA-S-P₂C-OT2) | | |
| NP thioether linked lipidated | 3,897 | 3,899.04 |
| construct | | |
| (NP-S-P₂C-OT2) | | |
| PA disulphide linked non- | 3,288.00 | 3,291.99 |
| lipidated construct | | |
| (PA-SS-OT2) | | |
| NP disulphide linked non- | 3,128.70 | 3,131.75 |
| lipidated construct | | |
| (NP-SS-OT2) | | |
| PA disulphide linked | 4,117.20 | 4,120.11 |
| lipidated construct | | |
| (PA-SS-P₂C-OT2) | | |
| NP disulphide linked | 3,957.90 | 3,960.58 |
| lipidated construct | | |
| (NP-SS-P₂C-OT2) | | |

¹′Detected using ESI-MS

### EXAMPLE 7

### Thioether-Linked Modular Constructs

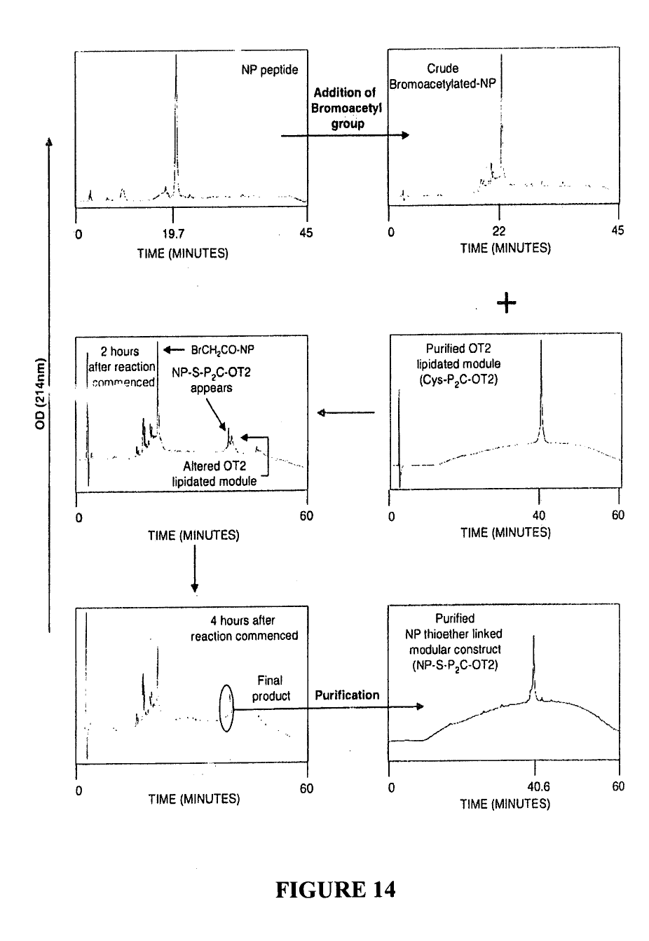

The first CTL epitope modification carried out involved the addition of a bromoacetyl group to the exposed N-terminal amino group of both the PA and NP epitopes while still attached to the solid phase support. This modification enabled the bromoacetyl-CTL epitope to form a thioether bond with the N-terminal thiol group of the OT2 lipidated or non-lipidated module (FIG. 5). Modification of either CTL epitope with bromoacetic acid was a rapid reaction, with complete bromoacetylation occurring within 30 minutes as confirmed by a TNBSA test. The modification was also accompanied by a shift in R_{T} of 2.2 minutes for the PA peptide (FIG. 13) and 2.3 minutes for the NP peptide (FIG. 14) and successful modification verified by the presence of the correct mass for both peptides (Table 5). Both bromoacetylated peptides were obtained in pure form following cleavage from the solid phase support and used for direct reaction with the OT2 modules without further purification.

RP-HPLC analysis was used to monitor the ligation reaction between either the bromoacetyl-PA or bromoacetyl-NP epitopes and the thiol-based OT2 lipidated module (FIG. 13 and FIG. 14, respectively). Approximately 5 minutes after initiating the reaction, the peak corresponding to the lipidated PA modular construct (R_{T} 40.3 minutes) is large and the peak corresponding to the OT2 lipidated module (R_{T} 40 minutes) is very small and almost undetectable. At 2 hours the chromatogram is very similar to the one obtained at 5 minutes with the peak corresponding to the thioether linked modular construct becoming slightly larger. This result indicated that the ligation between bromoacetyl-PA and the OT2 lipidated module is almost complete within 5 minutes.

In contrast to formation of the PA-based vaccine, the bromoacetyl-NP-based ligation was of low yield. Formation of an altered OT2 lipidated module was a significant side reaction. Initial reactions using 2-fold excess of the bromoacetyl-NP peptide generated this altered OT2 module as the main product. Increasing the excess of bromoacetyl-NP peptide to 10-fold improved the yield of the NP-based vaccine, although RP-HPLC analysis indicated that there was still a significant amount of the side reaction product present (FIG. 14). By using anaerobic conditions (blanketing the reaction vessel with nitrogen gas) there was not a decrease in the amount of side reaction product formed. Considering the similar ligation process used for both NP and PA it is likely that the NP sequence itself contributes to this side reaction.

Purification of all thioether linked modular constructs was successfully carried out using RP-HPLC following which all constructs eluted as single major peaks when analysed using RP-HPLC (final chromatogram, FIG. 13 and FIG. 14 for the PA and NP lipidated modular construct chromatograms respectively, data not shown for non-lipidated constructs). All species had the correct mass as determined by ESI-MS (Table 5).

### EXAMPLE 8

### Disulphide-Linked Modular Constructs

The CTL epitopes were first modified by coupling a cysteine residue to the exposed N-terminal amino group of the peptide sequence while still attached to the solid phase support. Following removal of the cysteinyl-CTL (Cys-CTL) from the solid phase support, the epitopes were reacted with 2,2′-dithiodipyridine (DTDP) in order to guarantee correct pairing of the Cys-CTL with the cysteine residue of the OT2 module constructs in subsequent ligation reactions (FIG. 6).

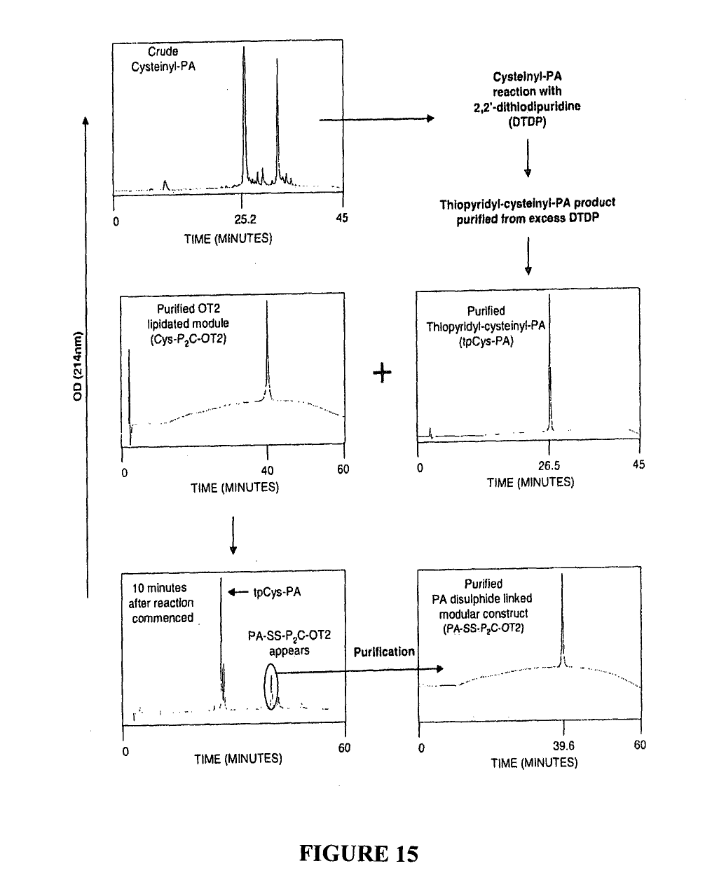

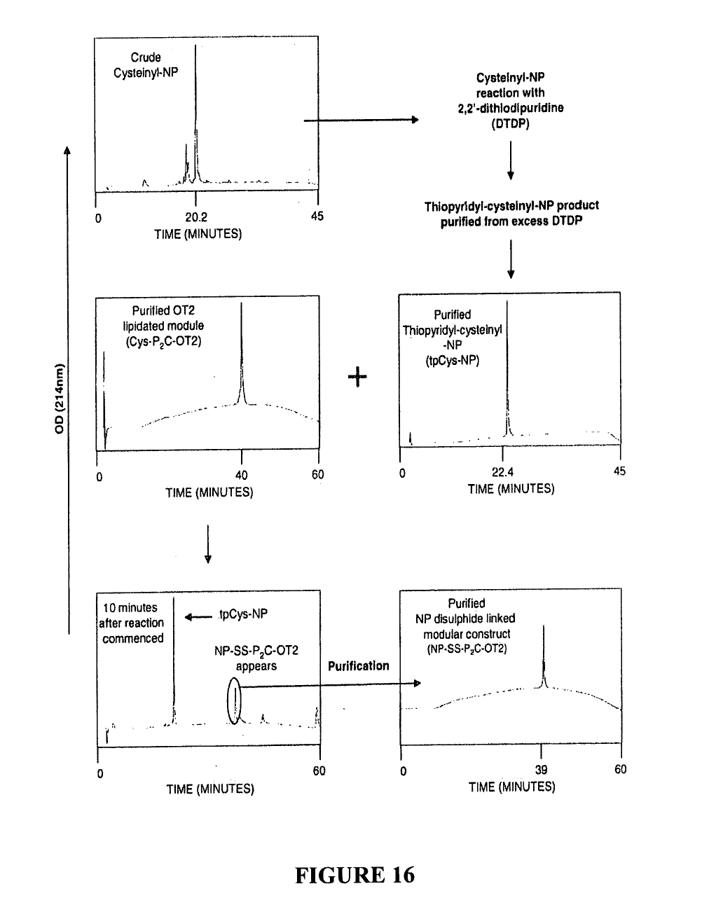

Formation of thiopyridyl-cysteinyl-CTL epitope (tpCys-CTL) was a rapid reaction. To prevent unreacted DTDP from reacting with the exposed thiol group present on the OT2 modules the tpCys-CTL peptide was isolated from the reaction mixture by RP-HPLC. Due to the similarity in R_{T} of the tpCys-PA peptide and DTDP, RP-HPLC separation was difficult, however this was overcome by reducing the amount of DTDP to a level at which excess DTDP in the reaction mixture was low and easily removed by RP-HPLC separation. The addition of the thiopyridyl (tp) group to the Cys-CTL epitopes caused the epitopes to elute slightly later with a RT shift from 25.2 minutes to 26.5, and 20.2 minutes to 22.4 minutes for the Cys-PA (FIG. 15) and Cys-NP (FIG. 16) peptides, respectively. The reactions with both Cys-PA and Cys-NP were fully complete after 30 minutes as determined by the shift in elution time by RP-HPLC and finding the correct mass using ESI-MS (Table 5).

The purified tpCys-PA and tpCys-NP epitopes were reacted with the OT2 modules to form the disulphide linked lipidated and non-lipidated modular constructs (FIG. 12). The ligation reaction to form the disulphide bond between the OT2 modules and the tpCys-CTL epitopes was very rapid. RP-HPLC analysis of both the tpCys-PA (FIG. 15) and tpCys-NP (FIG. 16) disulphide ligations with the OT2 lipidated module indicated that after 10 minutes the reaction was almost complete. In both cases, the chromatograms showed a major peak corresponding to that of the reaction product and a minor peak corresponding to the OT2 lipidated module. Following 1 hour of reaction time the peak corresponding to the OT2 lipidated module was absent in both reactions indicating complete reaction. Purification of all disulphide linked modular constructs (i.e. the PA lipidated and non-lipidated constructs and the NP lipidated and non-lipidated constructs) was successfully carried out using RP-HPLC, following which all constructs eluted as single major peaks when analysed using RP-HPLC (FIG. 15 and FIG. 16 for the PA and NP lipidated modular construct chromatograms respectively, (data not shown for non-lipidated constructs) and had the correct mass determined by ESI-MS (Table 5)).

### EXAMPLE 9

### Immunogenicity of the Modular Constructs—The Cellular Immune Response

The ability of the disulphide and thioether linked modular immunogens produced to induce a cellular response in C57BL/6 mice was determined. Groups of three mice were inoculated intranasally with either 25 nmol of each peptide immunogen in saline or infected with 10⁴ PFU of HKx31 virus. A mixture of 25 nmol of each the of PA and NP lipidated modular constructs was also included. To study the secondary response mice were either primed i.n. with 25 nmol of each peptide immunogen in saline or 10⁷ PFU of PR8 virus i.p. and 21 days later mice received a second identical dose of peptide immunogen or were challenged with 10⁴ PFU of x31 virus. Seven days following primary or secondary inoculation lungs were harvested and cells assayed for their ability to produce an antigen-specific response in an IFN-γ ICS assay.

### EXAMPLE 10

### Primary Response to Thioether-Based Constructs

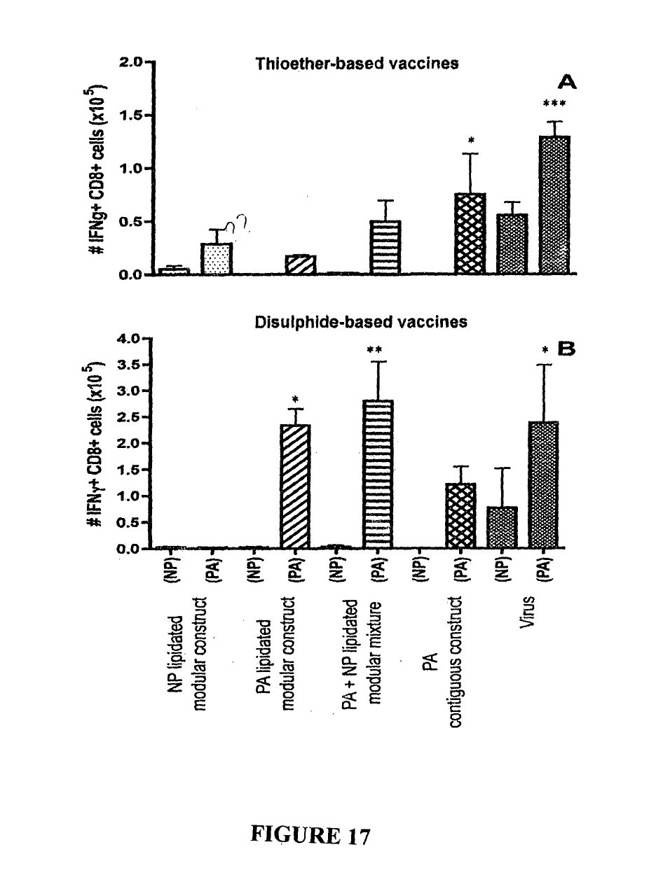

FIG. 17A shows the results from the IFN-γ ICS assay for the thioether modular constructs produced. The PA lipidated modular construct was able to elicit a PA₂₂₄₋₂₃₆ specific response, however, the magnitude was lower than that of the response induced following viral infection (p<0.001) or administration of the contiguously synthesised lipidated PA-based construct (p>0.05). The PA₂₂₄₋₂₃₆ specific response induced following viral infection was similar to that obtained after inoculation of the contiguously synthesised PA-based construct (p>0.05). No response was detected in the three groups of mice receiving PA or NP non-lipidated constructs or a mixture of the two, indicating the importance of the lipid moiety Pam₂Cys in inducing a cellular response.

The thioether linked NP lipidated modular construct induced a weak response, which was not significant when compared to the non-lipidated control (p>0.05). Unexpectantly, mice inoculated with the NP lipidated modular construct generated a slight but not significant (p>0.05) PA₂₂₄₋₂₃₆ specific response. Mice that received the PA and NP lipidated modular mixture generated a PA₂₂₄₋₂₃₆ specific response that was similar to mice that received the PA lipidated modular construct alone but no NP₃₆₆₋₃₇₄ specific response was detected. A significantly greater PA₂₂₄₋₂₃₆ specific than NP₃₆₆₋₃₇₄ specific response was detected with mice infected with virus (p<0.05).

### EXAMPLE 11

### Primary Response to the Disulphide-Based Constructs

The IFN-γ ICS assay results following inoculation with disulphide linked constructs are shown in FIG. 17B. The PA lipidated modular construct induced a PA₂₂₄₋₂₃₆ specific CD8⁺ response, which matched the response observed following viral infection (p>0.05). The PA lipidated modular construct also induced higher numbers of PA₂₂₄₋₂₃₆ specific IFN-γ⁺CD8⁺ cells than the PA contiguous construct although this difference was not significant (p>0.05). Again, no response was detected in the three groups of mice to which either PA or NP non-lipidated modular constructs or with a mixture of the two were administered.

The disulphide linked NP lipidated modular construct failed to induce a detectable NP₃₆₆₋₃₇₄ specific response. No PA₂₂₄₋₂₃₆ specific CD8⁺ response was detected in contrast to the result obtained when the thioether linked NP modular construct was given. A strong PA₂₂₄₋₂₃₆ specific response was obtained when a mixture of the PA and NP lipidated modular constructs were administered, which was as strong as that obtained following administration of the PA linked modular construct, however on NP₃₆₆₋₃₇₄ specific response was detected.

### EXAMPLE 12

### Secondary Response to Thioether-Based Constructs

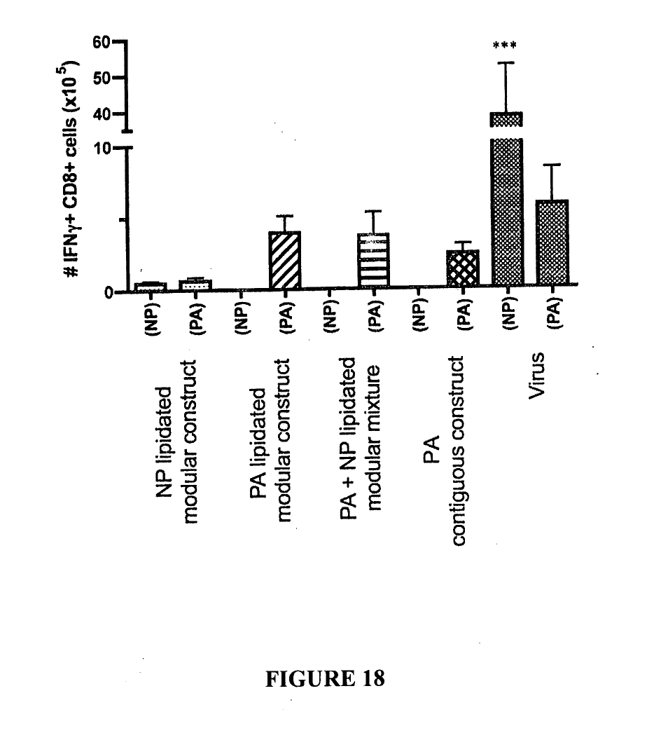

FIG. 18 displays the results following secondary inoculation of thioether linked modular constructs in mice. The PA thioether linked modular construct induced a higher number of PA₂₂₄₋₂₃₆ specific cells than the PA contiguous construct, although this was not significantly different (p>0.05). Interestingly, the PA linked modular construct also induced a similar PA₂₂₄₋₂₃₆ specific response to that induced following viral infection (p>0.05). All non-lipidated constructs were unable to induce PA₂₂₄₋₂₃₆ or NP₃₆₆₋₃₇₄ specific responses.

The NP lipidated modular construct induced a small NP₃₆₆₋₃₇₄ specific response and as observed in the primary response a slight PA₂₂₄₋₂₃₆ specific response was also observed. A strong PA₂₂₄₋₂₃₆ specific response was obtained when a mixture of the PA and NP lipidated modular constructs were administered to mice. This response was equivalent to that obtained following administration of the PA linked modular construct alone. No NP₃₆₆₋₃₇₄ specific response was detected in mice that received the lipidated modular mixture.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of said steps or features.

### BIBLIOGRAPHY

Alexander et al. Immunity 1:751-761, 1994

Barnden et al. Immunology and Cell Biology 76(1):34-40, 1998

Batzloff et al. Journal of Infectious Disease 194(3):325-30, 2006

BenMohamed et al. European Journal of Immunology 27(5):1242-53, 1997

BenMohamed et al. European Journal of Immunology 32(8):2274-81, 2002

BenMohamed et al. Immunology 106(1):113-21, 2002

BenMohamed et al. Infection and Immunity 72(8):4376-84, 2004

Berzofsky et al. J Clin Invest 88:876-884, 1991

Berzofsky, In: “The Year in Immunology, Vol. 2” page 151, Karger, Basel, 1986

Bodinier et al. Nature Med 6:707-710, 2000

Brown et al. J Virol 62(1):305-12, 1988

Calvo-Calle et al. Journal of Immunology 159(3):1362-73, 1997

Chua et al. Vaccine 25:92-101, 2007

Davenport et al. J Immunol 100(5):1139-40, 1968

Deliyarmis et al. European Journal of Immunology 36(3):770-8, 2006

Ghosh et al. Immunol 104:58-66, 2001

Goodman and Sercarz Ann Rev Immunol 1:465, 1983

Gosh et al. Immunology 104(1):58-66, 2001

Hancock and Batters by Analytical Biochemistry 71(1):260-4, 1976

International Patent Publication No. WO 00/46390

Jackson et al. Proc Natl Acad Sci USA 101(43):15440-5, 2004

Jackson et al. Virol 198:613-623, 1994

Jones et al. Xenobiotica 5:155, 1975

Livingstone and Fathman Ann Rev Immunol 5:477, 1987

Metzger et al. Int J Pept Protein Res 55:545, 1991

Metzger et al. J Pept Sci 1:184, 1995

Muhlradt et al. Infect Immun 66:4804, 1998

Muhlradt et al. J Exp Med 185:1951, 1997

Muller et al. Mol Immunol 32:37-47, 1995

Ogg et al. Curr Opin Immunol 10:393-396, 1998

Panina-Bordignon et al. Eur J Immun 19: 2237-2242, 1989

Partidos et al. J Gen Virol 71:2099-2105, 1990

Petrovsky et al. J Immunol Methods 186:37-46, 1995

Pialoux et al. Aids 15(1):1239-49, 2001

Robertson et al. Journal of Immunology 164(9):4706-12, 2000

Sacht et al. Eur J Immunol 28:4207, 1998

US Patent Application No. 0020018806

U.S. Pat. No. 4,196,265

Walker et al. Vaccine 25(41):7111-9, 2007

Wiesmuller et al. Hoppe Seylers Zur Physiol Chem 364:593, 1983

Winzler et al J Exp Med 185, 317, 1997

Zeng et al. J Pept Sci 2:66, 1996

Zeng et al. Journal of Peptide Science 2(1):66-72, 1996

Zeng et al. Vaccine 18(11-12):1031-9, 2000

1. A vaccine component comprising a T_{H} epitope, a lipid moiety and a linker wherein the T_{H} epitope is covalently linked to the lipid moiety via the linker and wherein the linker has a free reactive group.

2. The vaccine component of claim 1, wherein the linker is an amino acid or other tri-functional moiety.

3. The vaccine component of claim 2, wherein the amino acid is selected from the group consisting of aspartic acid, glutamic acid and analogs thereof.

4. The vaccine component of claim 2, wherein the amino acid is selected from the group consisting of lysine, ornithine, diaminopropionic acid, diaminobutyric acid, and analogs thereof.

5. The vaccine component of claim 4, wherein the linker is lysine, the T_{H} is covalently linked to the carboxyl group of the lysine, the lipid moiety is covalently linked to the ε-amino group of the lysine and the α-amino group of the lysine is the free reactive group.

6. The vaccine component of claim 4, wherein the linker is lysine, the T_{H} is covalently linked to the α-amino group of the lysine, the lipid moiety is covalently linked to the ε-amino group of the lysine and the carboxyl group of the lysine is the free reactive group.

7. The vaccine component of claim 4, wherein the linker is lysine, the T_{H} is covalently linked to the α-amino group of the lysine, the lipid moiety is covalently linked to the carboxyl group of the lysine and the ε-amino group of the lysine is the free reactive group.

8. The vaccine component of claim 1, wherein the lipid moiety is selected from the group consisting of palmitoyl, stearoyl and decanoyl.

9. The vaccine component of claim 1, wherein the lipid moiety is a molecule having a structure of Formula (I):

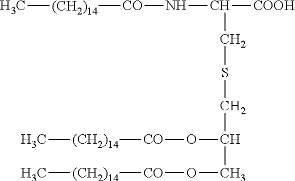

10. The vaccine component of claim 9, wherein the lipid moiety is N-palmitoyl-S-[2,3-bis(palmitoyloxy)propyl]cysteine.

11. The vaccine component of claim 1, wherein the lipid moiety is a molecule having a structure of Formula (II):

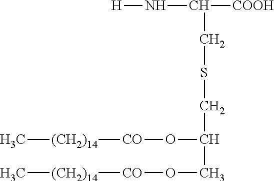

12. The vaccine component of claim 11, wherein the lipid moiety is S-[2,3-bis(palmitoyloxy)propyl]cysteine.

13. The vaccine component of claim 1, wherein the lipid moiety is a molecule having a structure of Formula (III): 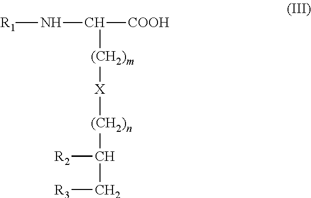
wherein:
(i) X is selected from the group consisting of sulfur, oxygen, disulfide (—S—S—), and methylene (—CH₂—), and amino (—NH—);
(ii) m is an integer being 0, 1 or 2;
(iii) n is an integer from 0 to 5;
(iv) R₁ is selected from the group consisting of hydrogen, carbonyl (—CO—), and R′—CO-wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group;
(v) R₂ is selected from the group consisting of R—CO—O—, R—O—, R—O—CO—, R′—NH—CO—, and R—CO—NH—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group; and
(vi) R₃ is selected from the group consisting of R—CO—O—, R′—O—, R′—O—CO—, R′—NH—CO—, and R—CO—NH—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group and wherein each of R₁, R₂ and R₃ is the same or different.

14. The vaccine component of claim 13, wherein the lipid moiety is a chiral molecule, wherein the carbon atoms directly or indirectly covalently bound to integers R₁ and R₂ are asymmetric dextrorotatory or levorotatory configuration.

15. The vaccine component of claim 13, wherein X is sulphur; m and n are both 1; R₁ is selected from the group consisting of hydrogen, and R′—CO—, wherein R′ is an alkyl group having 7 to 25 carbon atoms; and R₂ and R₃ are selected from the group consisting of R′—CO—O—, R′—O—, R′—O—CO—, R′—NH—CO—, and R—CO—NH—, wherein R′ is an alkyl group having 7 to 25 carbon atoms.

16. The vaccine component of claim 13, wherein R′ is selected from the group consisting of: palmitoyl, myristoyl, stearyl and decanol.

17. The vaccine component of claim 1, wherein the lipid moiety is a molecule having a structure of Formula (IV):

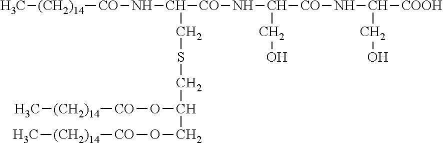

18. The vaccine component of claim 1, wherein the lipid moiety is a molecule having a structure of Formula (V):

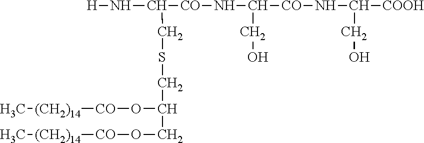

19. The vaccine component of claim 1, wherein the T_{H} epitope comprises an amino acid sequence selected from list consisting of SEQ ID NO:1, SEQ ID NO:2 and SEQ ID NO:3.

20. A method of generating a synthetic, self-adjuvanting lipopeptide vaccine construct, the method comprising chemical ligating a target epitope to a free reactive group on a linker to which a T_{H} epitope and a lipid moiety are covalently joined.

21. A method of producing a vaccine, the method comprising reacting the vaccine component of [any one of claims 1 to 19] claim 1 with an antigenic moiety comprising a CTL epitope and/or a B-cell epitope such that the antigenic moiety reacts with the free reactive group of the linker.
